(19) United States
(12) Patent Application Publication
(10) Pub. No.: US 2003/0027777 A1
(43) Pub. Date: Feb. 6, 2003

(54) Methods for enhancing animal growth and cell proliferation by elimination of the cyclin-dependent kinase inhibitor function of p27Kip1

(76) Inventors: Andrew Koff, New York, NY (US); Hiroaki Kiyokawa, Chicago, IL (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/038,060

(22) Filed: Jan. 4, 2002

Related U.S. Application Data
(63) Continuation of application No. 08/973,823, filed on Dec. 10, 1997, now abandoned.
Application No. 08/973,823 is a 371 of international application No. PCT/US97/05921, filed on Apr. 10, 1997.
(60) Provisional application No. 60/015,097, filed on Apr. 10, 1996.

(51) Int. Cl. A61K 48/00; C12N 15/85

(52) U.S. Cl. 514/44; 424/93.21; 435/455

(57) ABSTRACT

This invention provides a recombinant non-human animal lacking the cyclin-dependent kinase inhibitor function of p27^{Kip1} and the method for producing the same. This invention also provides a method for increasing the proliferation of the thymic T-cells by treating the thymic T-cells to eliminate the cyclin-dependent kinase inhibition function of p27^{Kip1}. This invention also provides a method for increasing the proliferation of hematopoietic cells which comprises treating the hematopoietic cells to eliminate the cyclin-dependent kinase inhibitor function of p27^{Kip1}, thereby increasing the proliferation of the hematopoietic cells. This invention further provides a method for alleviating symptoms of an AIDS patient comprising steps of: a) collecting the lymphocytes or other cells from an AIDS patient; b) treating the collected cells to eliminate the cyclin-dependent kinase inhibition function of p27^{Kip1}; and c) re-introducing the treated cells to the AIDS patient.

This application is a continuation of U.S. Ser. No. 08/973,823 (Dec. 10, 1997), which is a 371 of PCT/US97/05921 (Apr. 10, 1997), and which claims the benefit of U.S. Ser. No. 60/015,097 (Apr. 10, 1996).

The invention disclosed herein was supported in part by the National Institutes of Health Grant, GM52597. Accordingly, the U.S. Government has certain rights in the invention.

Throughout this application, various publications are referenced by author and date. Full citations for these publications may be found listed alphabetically at the end of the specification immediately preceding the claims. The disclosures of these publications in their entireties are hereby incorporated by reference into this application in order to more fully describe the state of the art as known to those skilled therein as of the date of the invention described and claimed herein.

### BACKGROUND OF THE INVENTION

Activation of cyclin-dependent kinases (CDKs) regulates progression through critical transitions in the cell cycle (Sherr, 1993). CDKs minimally contain a catalytic subunit, the CDK, and a regulatory subunit, a cyclin. Two distinct CDKs with overlapping functions, CDK4 or CDK6 and CDK2, control entry into S-phase (Ohtsubo and Roberts, 1993; Quelle et al., 1993, Resnitzky et al., 1994; Resnitzky and Reed, 1995). The activation state of these kinases ultimately determines the fate of cell proliferation during G1; when active cells enter S-phase. Inactivation of either kinase leads to cessation of proliferation and withdrawal from mitotic cycle. The activity of either CDK4/6, CDK2, or both kinases is absent in growth arrested cells. CDC2 kinase activity appears after the G1/S transition and is necessary for the G2/M transition.

G1 CDKs integrate mitogenic and anti-mitogenic signals that regulate progression through G1 to a point where further cell cycle progression continues autonomously (Pardee, 1989). This implies a capacity to regulate CDKs in response to external factors (Peters and Herskowitz 1995) The activation of CDKs is subject to multiple levels of regulation: the synthesis of the cyclin and CDK (Ewen et al., 1993; Geng and Weinberg, 1993; Tanquay and Chiles, 1994), the assembly of these proteins into complexes (Serrano et al., 1993; Guan et al., 1996), the activation of these complexes (Koff et al., 1993), and the ability of these complexes to phosphorylate substrates (Gyuris et al., 1993; Harper et al., 1993; Dulic et al., 1994; Kato et al., 1994; Polyak et al., 1994); Toyoshima and Hunter, 1994; Hannon and Beach, 1995; Lee et al., 1995). Recent work has identified two families of proteins that act as negative regulators of G1 CDKs, Inks and Cip/Kips (hereafter called Kips). Inks specifically target the CDK4 and CDK6 kinases and bind to these proteins preventing their interaction with cyclin D (Serrano et al., 1993; Guan et al., 1994; Hannon and Beach, 1994; Guan et al., 1996). In contrast, Kips bind preferentially to cyclin/CDK complexes and either prevents their activation by the CDK-activating kinase (Koff et al., 1993), or inhibit their kinase activity (Polyak et al., 1994b; Slingerland et al., 1994; Toyoshima and Hunter, 1994; Lee et al. 1995; Matsuoka et al., 1995). In addition, Kips, unlike Inks, are promiscuous and interact with most G1 CDKs (Sherr and Roberts, 1995).

At present, there are three members of the Kip family: p21 (Gu et al., 1993; Harper et al., 1993; Xiong et al., 1993), p27 (Polayk et al., 1994b; Toyoshima and Hunter, 1994), and p57 (Lee et al., 1995; Matsuoka et al., 1995). These proteins contain a conserved domain that is both necessary and sufficient for cyclin/CDK interaction and inhibition (Nakanishi et al., 1995). In proliferating cells, p21 and p27 associate with active CDKs (Zhang et al. 1994; Soos et al., 1996). In vitro reconstitution experiments indicate a requirement for the association of multiple molecules of Kip with a cyclin/CDK complex for complete inhibition of kinase activity (Harper et al., 1995). Amino acids 17-to-71 of p21 contain the cyclin/CDK-binding/inhibition domain (Nakanishi et al., 1995), and can be further divided into three sub domains: A, B and M (Chen et al., 1996). Peptides composed of sequences from these domains, either separately or together, are not capable of inhibition (Chen et al., 1996). Similar homologous domains can be postulated in p27 within amino acids 28-to-79: 26-51 (domain A), 69-88 (domain B), and 37-56 (domain M). Deletion analysis and site-directed mutagenesis have established the importance of the putative A and B domains (Polyak et al., 1994b; Luo et al., 1995). The function of the remaining region of p27, amino acid 92-197, is unknown at this time.

The properties of p27 suggest that it might have an important role regulating entry into and exit from the mitotic cycle. First, there is correlation between growth arrest and the expression of p27. In different cell lines exposed to various anti-mitogenic conditions, there is a correlation between withdrawal from the riitotic cycle and an increase in the amount of p27/CDK2 complex (Koff and Polyak, 1995). The increase in amount of p27/CDK2 complex occurs through at least one of three mechanisms dependent on the cell type and the condition leading to growth arrest: release of p27 from a sequestered state in cyclin D/CDK4 complexes (Polyak et al., 1994); Reynisdotter et al., 1995), increase in the transcription of p27 (Liu et al., 1996), or increase in the stability of p27 protein (Pagano et al., 1995). Second, ectopic expression of p27 cDNA is sufficient to induce G1 arrest (Polyak et al., 1994b; Toyoshima and Hunter 1994), and in some cells induce differentiation phenotypes (Kranenberg et al., 1995; Liu et al., 1996), presumably by targeting G1 cyclin-dependent kinase activity. Third, antisense vectors targeted to p27 mRNA increase the fraction of cells in S-phase (Coats et al., 1996). These properties suggest that p27 might function to establish an inhibitory threshold which G1 CDKs must surpass before activation and entry into S-phase.

### SUMMARY OF THE INVENTION

This invention provides a recombinant non-human animal lacking the cyclin-dependent kinase inhibitor function of p27^{Kip1}.

The recombinant non-human animal may be made by altering the gene encoding p27^{Kip1} and introducing the altered gene into the genome of an animal. The alteration comprises addition, deletion or mutation of at least one nucleotide of the gene which encodes p27^{Kip1}. Part or all of the gene encoding for p27^{Kip1} may be deleted and replaced by at least one selectable marker gene.

This invention provides a recombinant non-human animal having an increase in the production of thymocytes as compared with the animals having the cyclin-dependent kinase inhibitor function of p27^{Kip1}.

This invention provides a method to produce recombinant non-human animal lacking the cyclin-dependent kinase inhibitor function p27^{Kip1}. The method comprises altering the gene encoding p27^{Kip1}, introducing the altered gene into the genome of the animal, identifying the altered gene encoding p27^{Kip1} carrying animals and interbreeding of the altered gene carrying animal to generate p27^{Kip1} deficient animal.

In an embodiment, the gene encoding p27^{Kip1} is altered by insertion of a selection marker gene and a negatively selectable marker gene is then inserted adjacent to the altered gene whereby the distance between the marker gene and the altered gene is sufficient to carry homologous recombination. This construct is introduced into embryonic stem cells and positively select the marker which alters the gene encoding p27^{Kip1} and negatively select the inserted adjacent marker. This positive and negative selection scheme will ensure that the altered gene encoding p27^{Kip1} will integrate to the correct locus in the genome of the animal.

This invention further provides that the altered stem cells can be introduced by microinjection of the altered embryonic stem cell line to blastocysts.

The invention further provides a method for increasing the proliferation of thymic T-cells in an animal by treating the animal to eliminate the cyclin-dependent kinase inhibitor function of p27^{Kip1}. The treatment may be performed by exposing the animal with different agents capable of inhibiting the cyclin-dependent kinase inhibitor function of the p27^{Kip1}. In an embodiment, the agent is an antibody or a portion of an antibody directed against p27 protein.

This invention also provides a method for increasing the proliferation of hematopoietic cells which comprises treating the hematopoietic cells to eliminate the cyclin-dependent kinase inhibitor function of p27^{Kip1}, thereby increasing the proliferation of the hematopoietic cells.

This invention also provides a method for increasing the amount of hematopoietic cells for bone marrow transplantation between a donor and a recipient comprising steps of: a) obtaining the bone marrow cells from the donor; b) treating the bone marrow cells to eliminate the cyclin-dependent kinase inhibitor function of p27^{Kip1}; c) introducing the treated cells to the recipient.

The invention also provides a method for alleviating symptoms of AIDS patients which comprises (a) collecting peripheral lymphocytes from the patient, and (b) treating the collected lymphocytes such that after the treated lymphocytes will lack the cyclin-dependent inhibitor function of p27^{Kip1}. In an embodiment of the preceding method, only T-cells are treated. In another embodiment, only helper T-cells are treated.

This invention also provides a method for alleviating symptoms of AIDS patients comprising, (a) collecting multipotent cells from an AIDS patient, and (b) treating the multipotent cells with an agent to eliminate the cyclin-dependent kinase inhibition function of p27^{Kip1}; and, (c) introducing the treated cells to the same AIDS patient.

Finally this invention provides a method for increasing the efficacy of a cytokine to a subject comprising administering to the subject an effective amount of the cytokine in the presence of an agent which is capable of inhibiting the cyclin-dependent kinase inhibitor function of p27^{Kip1}. The cytokines which may be used in this invention include but are not limited to GM-CSF, G-CSF erythropoietin, interleukins, interferons, megakaryocyte derived growth factors and stem cell factors.

### BRIEF DESCRIPTION OF THE FIGURES

FIG. 1: Targeted insertion of the p27 gene produces an amino-terminal truncated protein that does not inhibit G1-CDKs.

(A) Restriction map of the mouse p27 gene, the targeting vector, and the structure of the locus following recombination. The targeting construct contains a 6 kb genomic sequence encompassing both coding exons of p27 with a neo cassette introduced into the Sma I site at amino acid 42. Transcriptional direction of the neo gene is shown. The thymidine kinase gene (TK) is indicated to the 3′ end of the genomic sequences. Homologous recombination within the genomic sequence introduces the neo gene and eliminates the TK gene (Mansour et al., 1988). Neo gene insertion will introduce an Eco RI site. Thus a single probe to the 5′ end of the genomic sequences can be used to identify recombinant alleles at 4.0 kb and wild-type alleles at 7.5 kb.

(B) Southern blot analysis of DNA from mice obtained by intercross breeding of heterozygous mice. Mice from intercross breeding were genotyped using tail DNA digested with Eco RI prior to electrophoresis using the probe in panel A, and we determined the presence or absence of the 7.5 kb wild-type allele and the 4.0 kb mutant allele.

(C) Analysis of p27 protein products in p27^{+/+} and p27^{-/-} mouse embryonic fibroblasts. Immunoprecipitates of p27 or whole cell lysates were subjected to SDS-PAGE and immunoblotted with the antibodies described to the left of the panels. Antibody abbreviations are: p27-carboxyl terminal specific antibody (αp27-CT), p27-amino terminal specific antibody (αp27-NT), CDK2 and CDK4 carboxyl specific antibodies (αCDK2 and αCDK4, respectively), rabbit anti mouse Ig (RAM). Markers and the migration of the Ig heavy chain are indicated.

(D) Δ51 protein is not able to inhibit G1-CDK activity. Lysates were prepared from Sf9 cells coinfected with the baculoviruses expressing cyclin and CDK subunits indicated above each graph. Equal amounts (30 nM) of either p27 (black) or Δ51 (open) were added and kinase activity measured, relative to a control (hatched) where neither protein was added. The kinase activity was determined on a GST-Rb substrate and plotted as percentage of the control.

FIG. 2 Enhanced growth of p27^{-/-} mice.

(A) Representative picture of 8 week old mice derived from intercross breeding of p27 heterozygous mice. Genotypes are indicated on the left.

(B) The size of mice is a function of p27 gene copy number. We conducted a retrospective analysis of weight using 252 mice obtained by intercross breeding in our colony between April and November 1995. No mouse was used twice for this analysis. In the graph, the mean weight of mice is represented by bars. p27+/+, filled bars; p27+/−, stippled bars; p27−/−, open bars.

The actual weights of individual mice are indicated by the gray dots. The age (d, day; w, week), genotype, numbers of mice, range of weights, mean and standard deviation are indicated. There is a statistically significant difference between the p27^{+/+} and p27^{-/-} groups at 8 weeks (t=−6.110, p<0.01).

(C) Representative growth curve of two wild-type and p27^{-/-} littermates that were approximately the same weight within the first two weeks post-partum. Males are represented by the solid line and females by the broken line. p27+/+, filled squares and circles; p27−/−, open squares and circles.

FIG. 3 There is a correlation between expression of p27 protein in an organ and the increase in weight of the organ following p27 gene disruption.

(A) Relative organ weight in p27^{-/-} mice. Organs were dissected and weighed; results are expressed as weight relative to weight of controls from age and sex matched wild-type mice (mean±SEM, n=5).

(B) The same organs were homogenized and proteins extracted by sonication. Following SDS-PAGE the amount of p27 was determined by immunoblot using carboxyl-specific p27 antibodies. The top panel is from p27^{+/+} mice and the bottom panel from p27^{-/-} mice. Molecular weight is indicated to the right of each panel.

FIG. 4 Intermediate lobe hyperplasia in p27^{-/-} mice.

(A) p27^{-/-} mice have enlarged pituitaries which can be attributed to selective hyperplasia of the intermediate lobe. A composite picture comparing hemotoxylin and eosin stained pituitary sections (top) from an eleven week old p27^{+/+} (left), 11 week old p27^{-/-} (middle) and a 30 week old p27^{-/-} (right) mouse. p, posterior lobe; i, intermediate lobe; a, anterior lobe. The intermediate lobe of 11 week old p27^{-/-} mice is increased in size relative to the p27^{+/+} controls with little change in cellular composition. In 30 week old p27^{-/-} mice the intermediate lobe is clearly hyperplastic with a nodular appearance resulting in the disruption of the posterior lobe tissue. In both p27^{-/-} mice the anterior lobe cells are compressed relative to the wildtype controls, however, the cell number does not appear to be altered. 45×magnification.

(B) Intermediate lobe cells of p27^{-/-} mice stain positively for POMC-derived peptides. Composite picture of pituitary sections (bottom) stained by avidin-biotin horseradish peroxidase immunocytochemistry for POMC-derived peptides. p, posterior lobe; i, intermediate lobe; a, anterior lobe. In 11 week old p27^{+/+} (left) and p27^{-/-} (middle) mice all intermediate lobe cells are uniformly stained while intermediate lobe cells of 30 week old p27^{-/-} mice (right) display isolated and heterogeneous staining. 114×magnification.

FIG. 5 Increased proliferation of thymocytes accounts for the increase in T-cell number.

(A) The proportion of thymocytes in S-phase is increased in p27^{-/-} mice. Mice were labeled with BrdU for two hours and incorporation into thymocytes was determined by anti-BrdU antibody staining. Sections of thymus isolated from either p27^{+/+} (panels a-c) or p27^{-/-} (panels d-f) mice are shown on the left and right respectively. (a, d) 40×magnification of the medullar region. The scale corresponds to 100 microns (b, e) 20×magnification showing both cortical and medullar regions. The scale corresponds to 50 microns. (c, f) Same as panels (b) and (e) but anti-BrdU antibody was omitted.

(B) Thymocytes of the p27^{-/-} mice are susceptible to apoptosis. An example of two independent experiments is shown. Thymocytes were isolated from mice and exposed to either 0.25 or 5 Gray (Gy) of irradiation or to 1 mM dexamethasone as indicated below each sample. Apoptotic cells were detected by the appearance of acridine orange stained condensed or fragmented chromatin and plotted as a percent of the total cells. p27+/+, filled bars; p27−/−, open bars.

FIG. 6 Estrus is perturbed in p27^{-/-} mice. Vaginal smears were taken daily from p27^{+/+} (top) or p27^{-/-} (bottom) mice and stained with hemotoxylin and eosin. Histology is an indicator of position in estrus (Nelson et al., 1982). p27^{+/+} mice complete estrus in four days (from left to right) passing through diestrus (6A), proestrus (6B), estrus (6C) and metestrus (6D), each in a single day. p27^{-/-} mice have a prolonged diestrus and estrus phase, and there is an increase in the amount of mucus in the smears of diestrus phase of the p27^{-/-} mice.

FIG. 7 p27^{-/-} mice are deficient in corpus luteum formation.

(A) There is a recipricol pattern of staining with p27 and BrdU specific antibodies in the ovaries of p27^{+/+} mice. Ovaries from mice labeled with bromodeoxyuridine were isolated, sectioned, and stained with antibodies against either p27, bromodeoxyuridine, or a rabbit immunoglobulin control antibody, as indicated below each panel. This antibody was detected with a secondary antibody conjugated to horseradish peroxidase. p27 protein is expressed in luteal cells. Granulosa cells incorporate BrdU.

(B) Ovaries of the p27^{-/-} mice lack the highly differentiated corpus luteum structure observed in controls. Composite picture comparing the ovaries of p27^{+/+} (top) and p27^{-/-} (bottom) mice by hemotoxylin and eosin staining using 5× and 20×magnification (left and right respectively). Mature follicles are present in both animals. CL, corpus luteum; Gr F, Graffian follicle.

FIG. 8 Relationship between thymocyte number and genotype of female mice at 5, 8 and 13 weeks. Filled squares, p27+/+; Open squares, p27−/−.

### DETAILED DESCRIPTION OF THE INVENTION

The present invention provides a recombinant, non-human animal which comprises functionally deficiency of the cyclin-dependent kinase inhibitor function of p27^{Kip1}. The functional deficiency describing the p27^{Kip1} may occur in the DNA, RNA or protein level such that functional gene product of p27^{Kip1} is either not produced or deficient.

As used herein, the term “recombinant animals” means animals produced by the recombinant DNA technologies. The animals produced are genetically altered such that the kinase inhibitor function of the p27^{Kip1} is eliminated. It is the intention of this invention to cover animals which are genetically altered by recombinant DNA technology such that p27^{Kip1} is either not produced or it is functionally deficient.

In an embodiment, such a deficiency is created by an alteration at the DNA level. The alteration may be an addition, deletion, mutation or combination thereof.

In another embodiment, the alteration is created by an insertion. The insertion takes place in the region responsible for the cyclin-dependent inhibitor function of p27^{Kip1}. The insertion may be achieved by inserting at least one selectable marker gene into the coding region. The selectable marker genes include but are not limited to neomycin resistant gene, thymidine kinase gene, adenine phophoribosyl transferase gene, hypoxanthine-guanine phosphoribosyl transferase gene, dihydrofolate reductase gene or a combination of more than one of the preceding genes or other selectable marker genes known to an ordinary skilled in the art. These selectable marker genes will express particular phenotypes under appropriate selective conditions.

In an embodiment, the recombinant non-human animal of this invention is generated by deleting part or all of the coding region of the gene encoding p27^{Kip1} and replaced by neomycin resistant gene.

Various drug resistance genes are examples of selectable marker genes. Some of the drug resistant genes are neomycin resistant gene and dihydrofolate reductase which can be selected by methotrexate. Other selectable marker include thymidine kinase gene, adenine phosphoribosyl transferase gene, hypoxanthine-guanine phosphoribosyl transferase gene. In a preferred embodiment, neomycin resistance gene is used for selection and to altered the gene encoding p27^{Kip1}.

The altered gene can be introduced either: (1) at an early developmental stage such that it is stably integrated into the germline and somatic cells of the animal. One embodiment would be microinjection into fertilized eggs (1 cell stage) and development of transgenic founder animals using standard transgenic technology or (2) after the animal is born in which case the transgene is only introduced into somatic cells. Several related embodiments would include, but not limited to, the uses of retroviruses, adenoviruses, asialoglycoprotein (ASO/PL/DNA) DNA complexes or liposomes to transfer the altered genes to somatic cells for the purpose of producing the claimed recombinant nonhuman animals. This embodiment is not limited to these methods but includes any method which transfers the altered gene to somatic cells for the purpose stated above.

The invention also provides a recombinant p27^{Kip1} deficient animals which show a significant increase in the number of granulocyte/macrophage progenitors and mixed pluripotent progenitors in the spleen and a significantly greater number of thymocytes as compared to the wild type mouse.

Although the below experiments discussed concern a recombinant p27^{Kip1} deficient mouse, it is clear that the recombinant animals of this invention may be of any animal species. Therefore, it is understood that the invention encompasses all animals.

In an embodiment, the animal is a mammal. In another embodiment, the animal is selected from a group consisting of rodent, cattle, pig and sheep. In a still another embodiment, the animal is a frog. It is recently identified that a p27^{Kip1} analog exist in Xenopus (Shou and Dunphy, 1996). In a separate embodiment, the animal is a fish. In a another separate embodiment, the animal is a Caenorhabditis elegans.

Another aspect of the invention involves a method to produce a recombinant p27^{Kip1} deficient non-human animal. Generally, such animal is produced by a) altering the gene encoding p27^{Kip1} so that the p27^{Kip1} gene product is functionally deficient, b) introducing the altered gene into the genome of the animal, c) identifying the altered gene carrying animals, and d) generating altered gene carrying animals which is p27^{Kip1} deficient.

The alteration of the gene encoding p27^{Kip1} comprises addition, deletion or mutation, or any other methods known to those skilled in the art. Alternatively, the DNA encoding p27^{Kip1} may be altered by methylation or the like to inactivate the gene expression.

In an embodiment, the gene encoding p27^{Kip1} is cloned in a plasmid and the alteration of the gene encoding p27^{Kip1} is done on the cloned DNA. Various plasmids well known to a skilled practitioner will serve this purpose.

The introduction of the altered gene encoding p27^{Kip1} into an animal comprises steps of (a) electroporating of the altered gene into embryonic stem cells, (b) culturing in vitro the treated embryonic stem cells, (c) selecting of the deficient p27^{Kip1} carrying embryonic stem cells and introducing the deficient p27^{Kip1} carrying embryonic stem cells to the blastocysts by microinjection. The blastocysts are introduced into the animal.

In an embodiment, the gene encoding p27^{Kip1} is isolated and cloned in a plasmid and then altered by insertion of a selection marker gene. A negatively selectable marker gene is then inserted adjacent to the altered gene whereby the distance between the marker gene and the altered gene is sufficient to carry out homologous recombination. The plasmid containing the altered gene is used to transform an embryonic stem cell line. The cells which incorporate the altered gene are positively selected for the marker in the gene encoding p27^{Kip1} locus and negatively selected for the inserted adjacent marker. The final selected altered embryonic stem cell line is microinjected into the blastocyst. Alternatively, the plasmid containing altered gene with the selection marker gene transformed stem cells are selected for the selectable marker phenotype.

In another embodiment, the negative selection marker is the thymidine kinase gene. The transfected stem cells are then positively selected with the expression of neomycin resistance phenotype and negatively selected with thymidine kinase phenotype.

The invention further provides a method for increasing the proliferation of thymic T-cells in an animal by treating the animal to eliminate the cyclin-dependent kinase inhibitor function of p27^{Kip1}. The treatment may be performed by contacting the animal with different agents capable of inhibiting the cyclin-dependent kinase inhibitor function of the p27^{Kip1}. In an embodiment, the agent is an antibody or a portion of an antibody directed against p27 protein. To facilitate the delivery of the some agent into the cells, the agents may be entrapped in liposomes for efficient delivery. Methods to make liposomes are well-known in the art.

This invention also provides a method for increasing the proliferation of T-cells which comprises treating the T-cells to eliminate the cyclin-dependent kinase inhibitor function of p27^{Kip1}, thereby increasing the proliferation of the T-cells.

This invention also provides a method for increasing the proliferation of hematopoietic cells which comprises treating the hematopoietic cells to eliminate the cyclin-dependent kinase inhibitor function of p27^{Kip1}, thereby increasing the proliferation of the hematopoietic cells.

This invention also provides a method for increasing the amount of hematopoietic cells for bone marrow transplantation between a donor and a recipient comprising steps of: a) obtaining the bone marrow cells from the donor; b) treating the bone marrow cells to eliminate the cyclin-dependent kinase inhibitor function of p27^{Kip1}; c) introducing the treated cells to the recipient.

The invention also provides a method for alleviating symptoms of AIDS patients which comprises (a) collecting peripheral lymphocytes from the patient, and (b) treating the collected lymphocytes such that after the treated lymphocytes will lack the cyclin-dependent inhibitor function of p27^{Kip1}. In an embodiment of the preceding method, only T-cells are treated. In another embodiment, only helper T-cells are treated.

This invention also provides a method for alleviating symptoms of AIDS patients comprising, (a) collecting multipotent cells from an AIDS patient, and (b) treating the multipotent cells with an agent to eliminate the cyclin-dependent kinase inhibition function of p27^{Kip1}; and, (c) introducing the treated cells to the same AIDS patient.

In an embodiment, the cells are treated with an agent. In a further embodiment, the agent is an antisense oligonucleotide capable of inhibiting the expression of p27^{Kip1} In a further embodiment, the agent is a vector containing a promoter operatively linked to a cDNA which encodes an antisense mRNA capable of inhibiting the expression of p27^{Kip1}.

The above-described cells including lymphocytes, thymic T-cells, helper T-cells, hematopoietic cells and bone marrow cells may be treated with linearized vector containing the gene encoding p27^{Kip1} which is disrupted by a selectable marker gene. Cells with incorporated the vector will be selected in an appropriate selection medium. The event of homologous recombination may be confirmed by the performing restriction enzyme digests.

In an embodiment, the treatment is performed by contacting the above-described cells with an oligonucleotide antisense encoding the region of p27^{Kip1} responsible for the cyclin-dependent kinase inhibitor function of p27^{Kip1}.

Antisense DNA or RNA which can hybridize with the gene encoding p27^{Kip1} may be introduced into the cells to hybridize with the p27^{Kip1} mRNA such that no translation can occur and therefore, no functional protein is produced. The methods for introduction of the antisense DNA or RNA into the cell are well known to a skilled practitioner. One method is to clone and express an antisense under a promoter such that a larger amount of antisense RNA against the mRNA encoding p27^{Kip1} will be produced. The antisense for p27^{Kip1} RNA produced will hybridize with the normal p27^{Kip1} mRNA and therefore, interfering with the expression of p27^{Kip1}.

In another embodiment, cells are treated with chemicals or small peptides capable of preventing p27^{Kip1} from associating with cyclin/CDK complex.

The invention also provides a method for alleviating the symptoms of AIDS patients, comprising administering to the patient an effective amount of an adenovirus or any suitable vector containing a promoter operatively linked to a cDNA which encodes an antisense mRNA capable of inhibiting the expression of p27^{Kip1}.

Finally this invention provides a method for increasing the efficacy of a cytokine to a subject comprising administering to the subject an effective amount of the cytokine in the presence of an agent which is capable of inhibiting the cyclin-dependent kinase inhibitor function of p27^{Kip1}. The cytokines which may be used in this invention include but are not limited to GM-CSF, G-CSF erythropoietin, interleukins, interferons, megakaryocyte derived growth factors and stem cell factors such as c-kit.

This invention is illustrated in the Experimental Details section which follows. These sections are set forth to aid in an understanding of the invention but are not intended to, and should not be construed to, limit in any way the invention as set forth in the claims which follow thereafter.

### FIRST SERIES OF EXPERIMENTS

### Experimental Details

### Experimental Procedures

Targeted Mutagenesis in the Mouse p27 Gene

The mouse p27 gene was isolated by screening a 129SV mouse genomic library (Stratagene) using the total coding region of mouse p27 cDNA as a probe. A 6.0 kbp Bam HI fragment that contained the entire coding sequence, exons I and II, was subcloned into a pBS vector. This construct was cleaved at a Sma I site located in codon 42 of exon 1 by partial digestion, and a 1.1 kb blunt-ended Xho I-Bam HI fragment encoding pMC-1neo polyA (Stratagene) was inserted. This construct was subsequently digested with Not I and Bam H1, and inserted a 1.2 kb HSV-TK gene cassette driven by the polyoma enhancer.

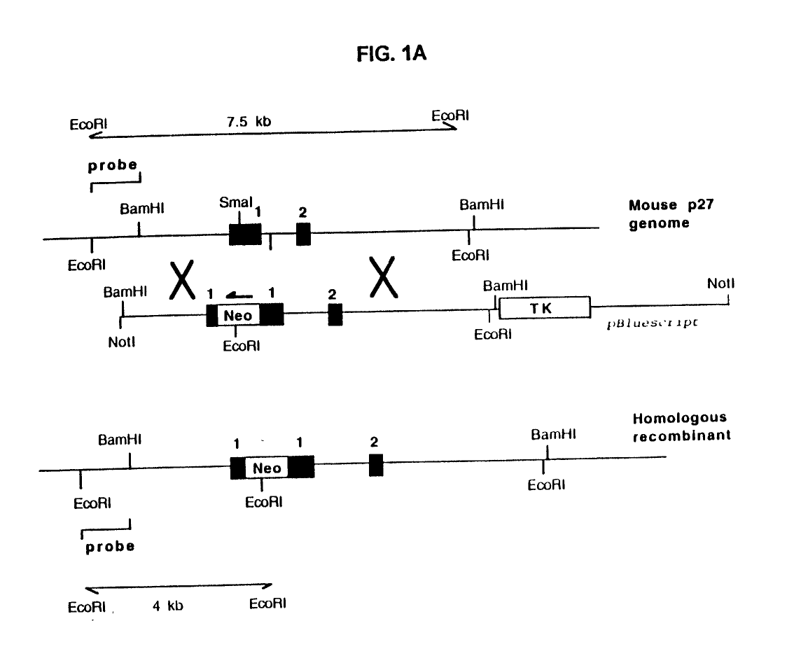

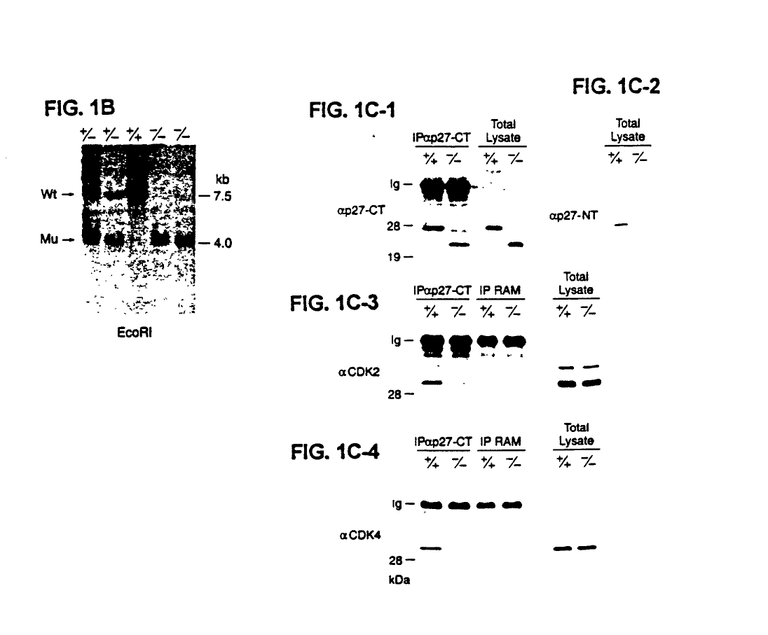

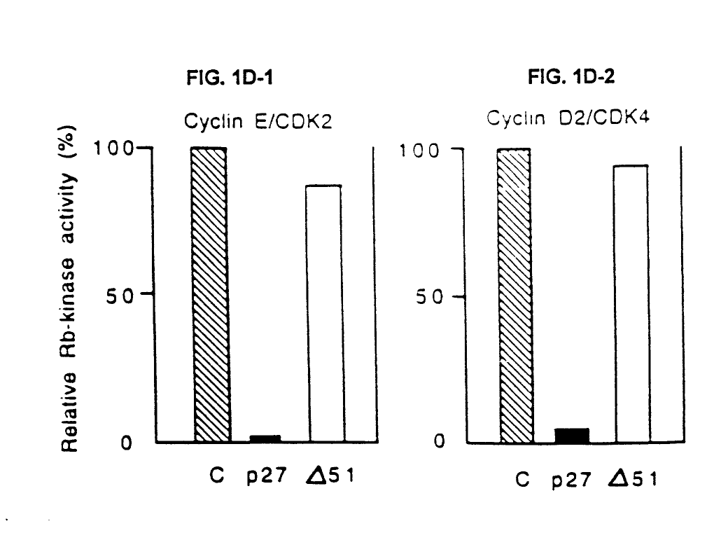

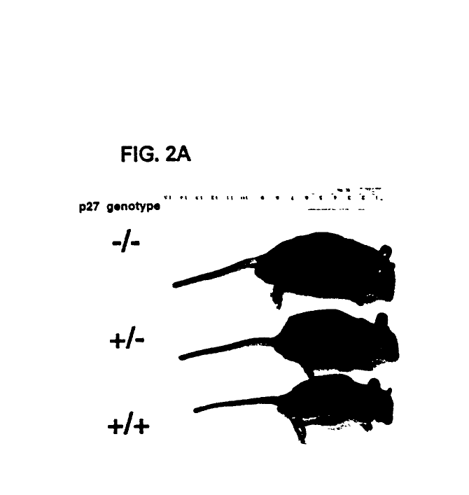

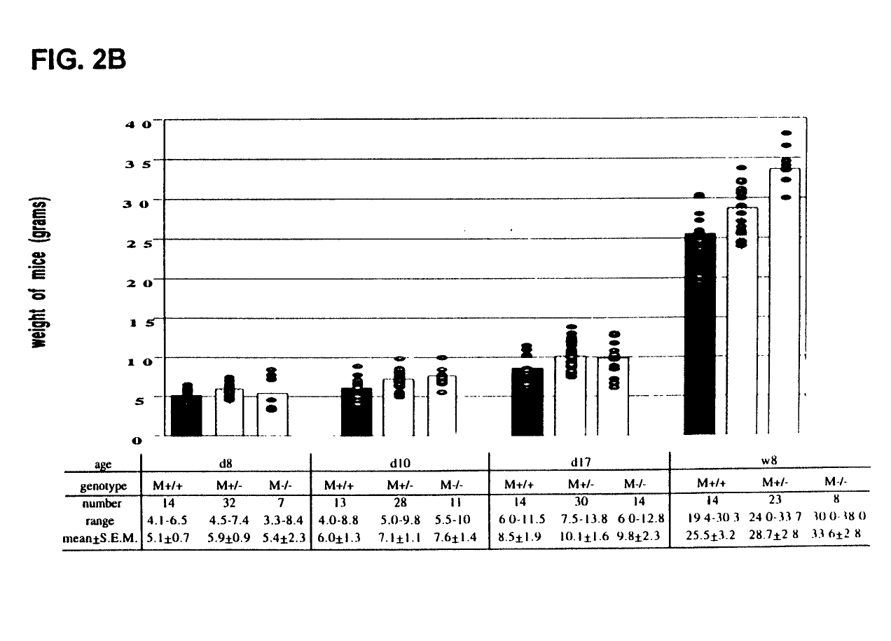

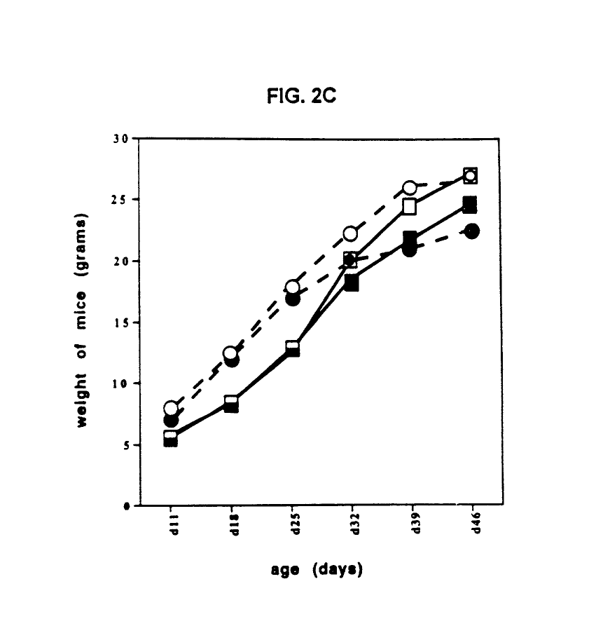

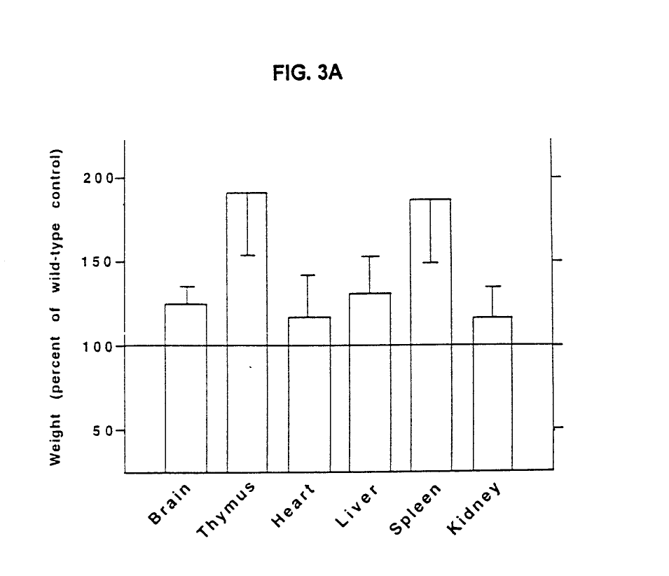

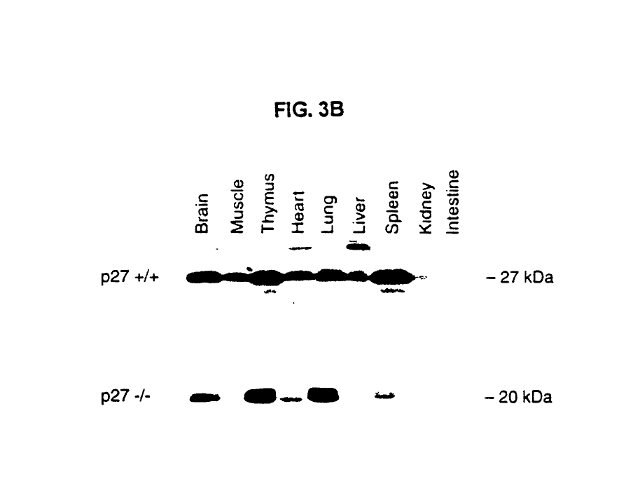

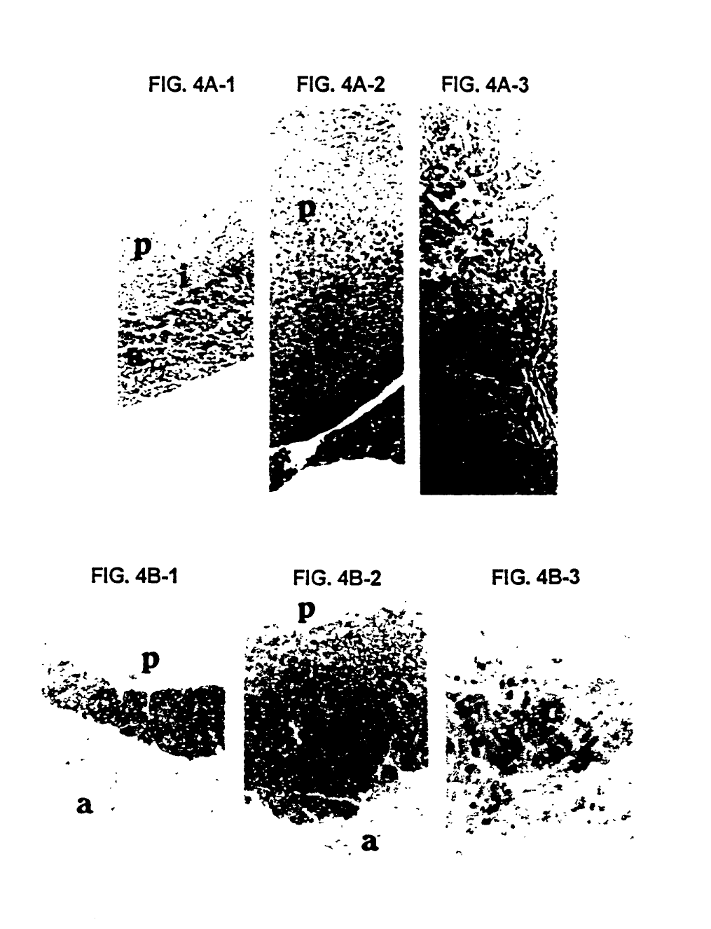

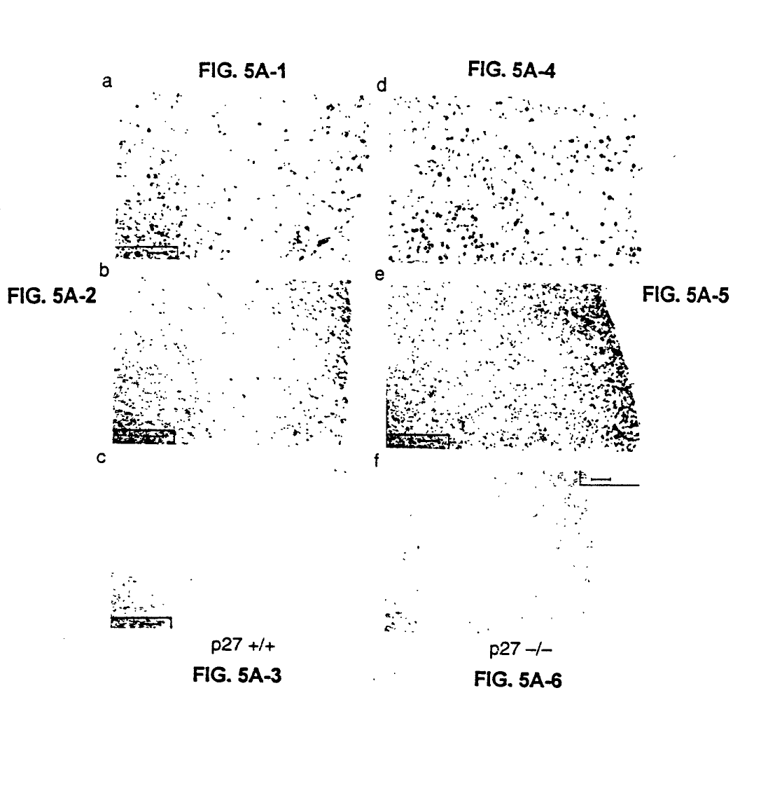

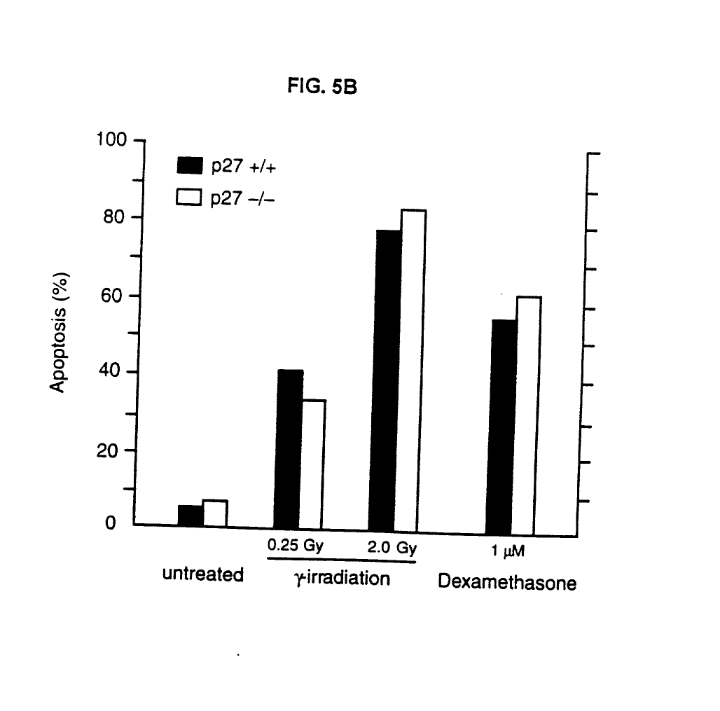

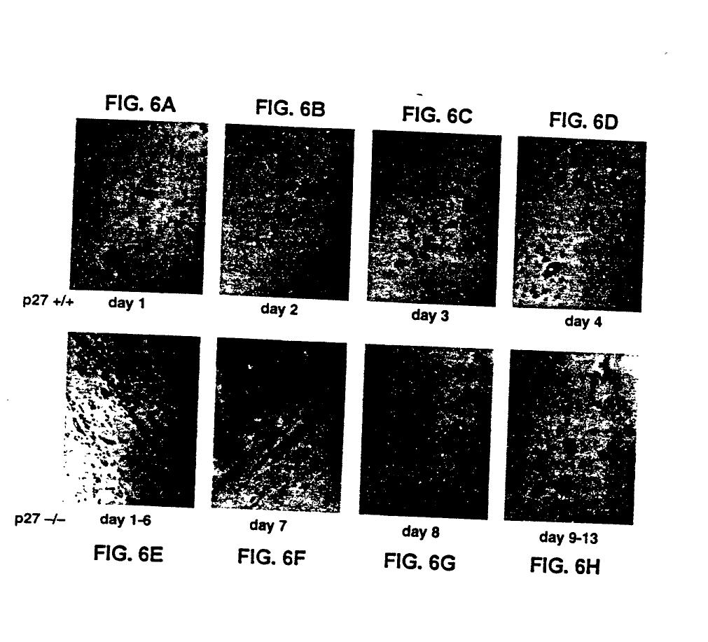

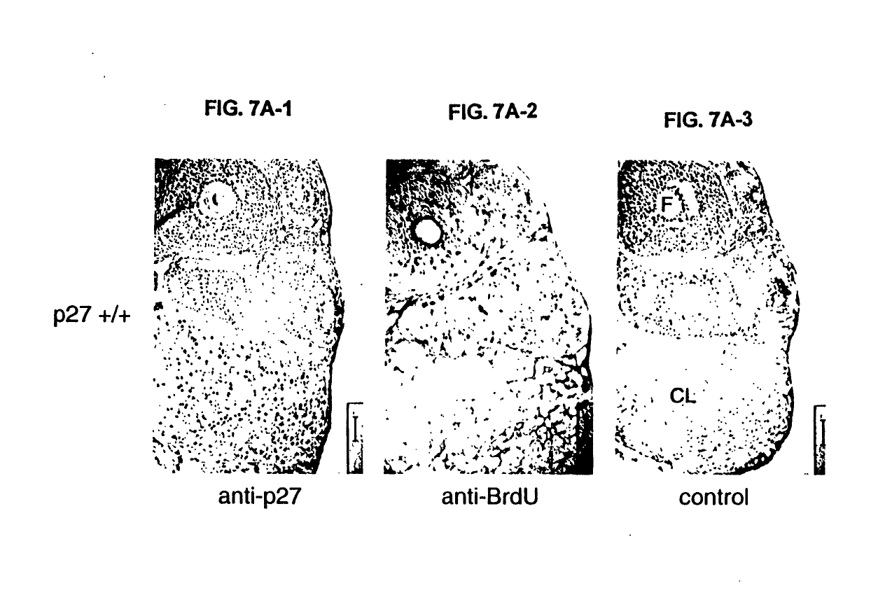

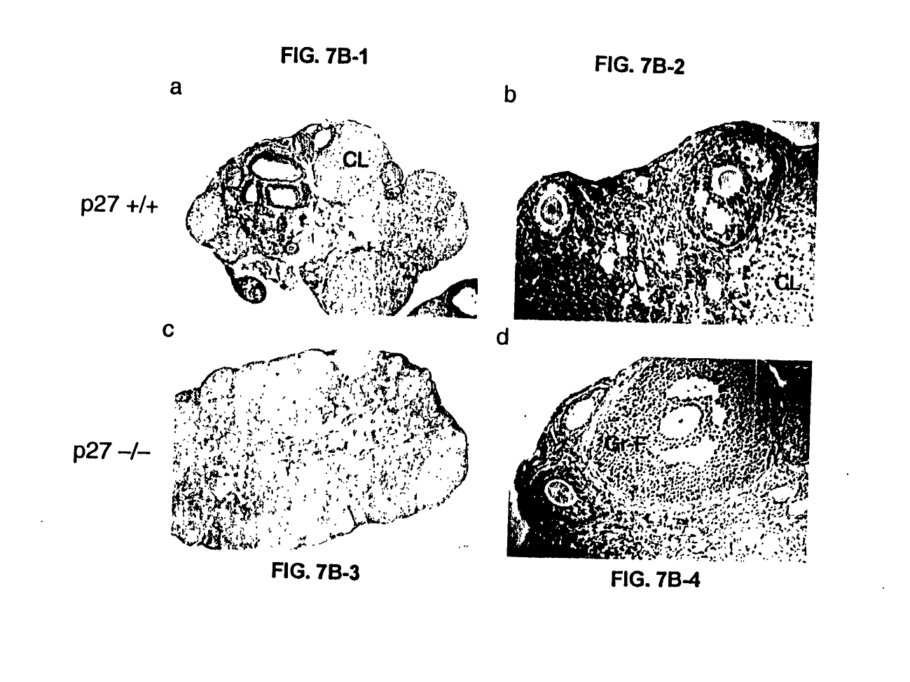

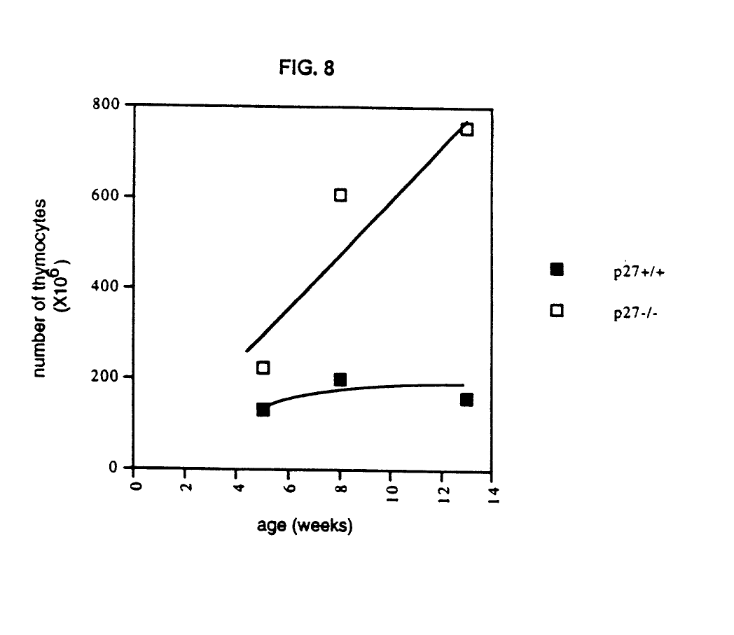

Not I-linearized targeting vector was introduced into CJ7 ES cells by electroporation (Swiatek and Gridley, 1993). Positively transfected cells were selected in 0.5 mg/ml G418 and 0.2 mM ganciclovir, and the surviving colonies were picked, expanded and analyzed with their genotype at the p27 locus by Southern blotting. Genomic DNA was digested with Eco RI. The 1.1-kb Eco RI-Bam HI fragment 5′ to the vector fragment was used as a probe as shown in FIG. 1. This probe hybridizes to a 7.5 kb Eco RI fragment from the wild-type allele, and a 4.0 kb Eco RI fragment from the homologous recombinant allele (FIG. 1B).

p27 heterozygous ES cells (10-15 cells per blastocyst) were microinjected into blastocyst stage C57BL/6 mouse embryos. Injected blastocysts were transplanted into uteri of pseudo pregnant C57BL/6 mice. Chimeric males were crossed to C57BL/6 females. Germline transmission of the injected ES cells monitored by detecting agouti mice among the F1 offspring, and subsequent Southern blotting as described above.

Cell Culture and Protein Analysis

Embryonic fibroblasts were prepared from embryos 13.5 days post coitus. Fibroblasts were cultured to confluence (passage 0) in a 75-cm² flask with Dulbecco's modified minimum essential medium (DMEM) supplemented with 10% fetal bovine serum (FBS), and sub-cultured to a density of 13,000 cells/cm² every 3-4 days. To prepare extracts, cells were lysed by sonication in Rb kinase lysis buffer containing Tween-20 (Matsushime et al., 1994), and protein content determined by Bradford assay.

For immunoblotting, 50 μg of extract was resolved on a 17.5% SDS-polyacrylamide gel and proteins blotted to Immobilon P (Millipore). p27 was detected with either affinity-purified antibody raised against a glutathione-S-transferase p27 fusion protein (GST-p27) (Soos et al., 1996) or a p27 carboxyl-terminal specific antibody (Santa Cruz Biotechnology) as described (Soos et al., 1996). For detection of CDK proteins in p27 immunoprecipitates, p27 was immunoprecipitated from 300 μg of extract and blotted with antibodies specific for the carboxyl terminus of CDK2 and CDK4 (Santa Cruz Biotechnology).

Preparation of Recombinant Proteins and Kinase Assays

Both His-tagged full-length (wild-type) and the His-tagged amino-terminus truncated form of p27 were expressed in bacteria transformed with the pET21a (Novagen). Proteins were purified by metal affinity chromatography as previously described (Polyak et al., 1994). Baculovirus encoding cyclin E and CDK2 were kindly provided by D. Morgan. Baculovirus for cyclin D2 and CDK4 were kindly provided by C. Sherr. Rb kinase experiments using baculovirus-encoded cyclins and CDKs were performed as described previously (Polyak et al., 1994b). Briefly, Sf9 insect cells were co-infected with viruses expressing either cyclin E and CDK2, or cyclin D2 and CDK4, and extracts were prepared 48 hours later by sonication in the Rb kinase reaction buffer containing 10 mg/ml aprotinin, 10 mg/ml leupeptin, 10 mg/ml soybean trypsin inhibitor and 1 mM phenyl-methylsulfonyl fluoride (PMSF). Clarified extracts were incubated at 30° C. for 30 minutes with [γ-³²P]ATP, GST-Rb fusion protein, and increasing amounts of wild-type or mutant p27 protein. Reactions were stopped by the addition of sample buffer and proteins separated on a 10% SDS-polyacrylamide gel. The incorporation of [γ³²P]ATP into GST-Rb fusion protein was measured by phosphoimager (Fuji).

X-ray Imaging of Mice

Mice were anesthetized by intraperitoneal injection of avertin, immobilized on a cassette containing XAR film (Kodak), and irradiated at 70 kV for 0.2 sec.

IGF-I Measurements

Serum was obtained by retro-orbital sinus puncture from a collection of littermate pairs, both male and female. Six pairs of animals were used for this analysis. Three non-littermate animals that were included and age matched. Serum from each animal was obtained at different ages and these sera (total number is 27) were divided into two groups, those collected from 6-10 week old mice and those collected from 11-30 week old mice. IGF-I was measured by a double antibody radioimmunoassay using recombinant hIGF-I (Genentech) and anti-hIGF-I serum UBK487 (National Hormone and Pituitary Program, NIH) after removal of binding proteins from a 5 μl aliqot of serum by means of a Sep-Pak C18 reverse phase cartridge (Millipore). After obtaining all the measurements from these mice, the amount of IGF1 in p27+/+ and p27−/− mice were compared in the following ways: all sex matched, age/sex matched, and all age matched. Two other comparisons that focused only on the age and sex of the animal and not the genotype were included: all females compared to all males, and all age matched. There were no statistically significant differences between any group except for the comparison of male and female mice regardless of genotype (t=2.044, p<0.05).

Histology

Tissues, with the exception of the pituitary, were fixed in 4% paraformaldehyde at 4° C. overnight, treated stepwise with 50% ethanol, 70% ethanol, 80% ethanol, 90% ethanol, histoclear, histoclear/paraffin (1:1), and paraffin, and sectioned at 4 microns. The sections were used for hematoxylin/eosin staining or immunohistochemistry as indicated in the legends to the figures.

To detect proliferating cells, mice were injected intraperitoneally (50 μg per g body weight) with bromodeoxyuridine (BrdU) (Morstyn et al., 1983). The mice were sacrificed 2 hours later. Thymus, spleen, and in females the ovaries were isolated and prepared the tissues for sectioning. The tissue sections were rehydrated and treated with 5 μg/ml proteinase K, 50 mM Tris-HCl (pH 7.5), and 5 mM EDTA at 37° C. for 10 minutes. The slides were washed with PBS and then acid-treated (1N HCl) for 10 minutes at 55° C. Slides were subsequently washed with PBS and immersed in PBS supplemented with 0.1% H₂O₂ for 15 minutes at room temperature. The samples were then blocked with the avidin/biotin blocking solution (Vector lab) and blocking buffer (10% horse serum, 2% BSA, 0.5% Tween 20) for 15 minutes. To detect BrdU applicants slides were incubated overnight at 4° C. in blocking buffer containing 6 μg/ml anti-BrdU monoclonal antibody (Boerhinger-Mannheim). The bound antibodies were subsequently detected with biotinylated secondary antibody (1:200 dilution) and a peroxidase-conjugated streptavidin according to the manufacture's instruction (Vectastain).

To detect p27 in sections from ovaries applicants used either affinity-purified antibody against GST-p27 (Soos et al., 1996), or the p27 carboxyl-terminal specific antibody. The specificity of these antibodies was confirmed by using antibodies pre-incubated with an excess of antigen before staining.

Thymocyte apoptosis was measured as described (Lowe et al., 1993; Clarke et al., 1993). For irradiation, thymocytes were exposed to γ-rays from a ¹³⁷Cs source at 0.1 Gy/min. Following irradiation cells were cultured in DMEM supplemented with 5% fetal calf serum for 8 hours. The cells were subsequently fixed to glass slides with acetic acid/ethanol solution (1:9) overnight. Fixed slides were treated sequentially with PBS, 0.1% Triton X-100, 0.08N HCl/0.15 M NaCl, and subsequently stained with 20 mM acridine orange (Sigma) in a phosphate-citric acid buffer (pH 6) containing 1 mM EDTA and 0.15 M NaCl. Cells were examined in a fluorescence microscope, and apoptosis determined by the presence of condensed or fragmented chromatin.

Pituitaries were removed at the time of necropsy from mice, both male and female, at 11 and 30 weeks of age and placed in 10% neutral buffered formalin and stored on 70% ethanol until processing. Tissues were paraffin embedded by standard methods, sectioned at 4 microns and stained with hematoxylin and eosin by standard methods.

Unstained paraffin sections of pituitaries were cleared in xylene and rehydrated in graded ethanol solutions. Sections were then treated sequentially with 0.3% H₂O₂ (30% stock solution) in methanol (30 minutes, room temperature (RT)), 1.5% normal goat serum (30 minutes, RT); rabbit-anti hACTH sera (1:10,000, NIH), or monkey-anti rat GH sera (1:50,000, LAF 82469) (24 hours, 4° C.); biotinylated goat-anti rabbit or goat-anti monkey IgG (1:200, 30 minutes, RT; Vector), Vectastain Elite ABC reagent (30 minute, RT; Vector); 0.05% 3,3′-diaminobenzidine tetrahydrochloride (DAB) in the presence of 0.05% H₂O₂ (5 minutes). Reagents were prepared in 0.05 M Tris buffered saline (TBS, pH 7.4) containing 0.1% Triton X-100, except for ABC and DAB which were diluted in TBS alone. Replacement of the primary immune serum with normal rabbit or monkey serum abolished staining. Specificity of each antisera was confirmed in a previous study by inhibition of staining when each sera was preabsorbed with the respective antigen (1 mg/ml dilute primary antibody) for 2 h at 37° C. before use in the immunocytochemical procedure.

Fluorescence-activated Cell Sorting

Single cell suspensions were made from the thymus and stained thymocytes as described previously (Wilson et al., 1988; Dudley et al., 1994). Data was collected on a Becton-Dickinson FACStar plus flow cytometer and analyzed using CellQuest 1.2 (Becton-Dickinson). CD4 antibody conjugated to Quantum red was obtained from Sigma. Antibodies to CD8α (phycoerythrin conjugated), CD25 (FITC conjugated), and CD44 (biotin conjugated) were obtained from Pharmingen. Streptavidin Texas Red was obtained from Vector.

Lymphocyte Activation

Spleen cells were obtained by pressing the spleen against the bottom of a tissue culture dish with a bent syringe needle. Cells were collected by centrifugation and resuspended them in 0.15M NH₄Cl, 1 mM KHCO₃, and 0.1 mM EDTA (pH 7.2) to lyse erythrocytes. The remaining cells were centrifuged through FBS, washed with PBS, and resuspended in RPMI 1640+10% FBS, 2 mM glutamine, non-essential amino acids, 1 mM sodium pyruvate, and 50 mM 2-mercaptoethanol. To eliminate B-lymphocytes anti-B220 antibody was added and incubated at 4° C. for 1 hour, and then added rabbit serum complement and continued the incubation for 45 minutes at 37° C. Cells that did not adhere to the flask during incubation were collected and washed extensively. By this procedure more than 80% of the resulting cells were T lymphocytes. Two×10⁵ viable cells were subcultured for 72 hours in 0.5 ml of RPMI +10% FBS containing various amounts of anti-CD3 antibody with or without 100 U/ml IL-2. [³H]thymidine (2 mCi/mmol, 1 mCi/sample) was added for the last 4 hours of the culture. The labeled cells were harvested and incorporated [³H]thymidine was measured by liquid scintillation counter.

### Experimental Results

Targeted Mutagenesis of the Mouse p27 Gene To investigate the function of p27 in mammalian development, the p27 coding sequence was disrupted by homologous recombination using a vector containing genomic p27 isolated from a 129Sv mouse DNA library (FIG. 1A). The mouse p27 gene is encoded by two exons divided within codon 159. To disrupt specifically the cyclin/CDK inhibition domain of p27, a neomycin-resistance (neo) gene cassette was inserted into codon 42, ligated a thymidine kinase (TK) gene cassette to the 3′-end of the genomic fragment, and selected CJ7 mouse embryonic stem (ES) cells (Swiatek and Gridley, 1993) expressing this cassette by positive-negative selection as described (Mansour et al., 1988). Out of approximately 800 colonies dual-resistant to G418 and ganciclovir, 80 clones were expanded and analyzed their genotypes by Southern blotting. Of these, 14 clones (18%) had a legitimate homologous recombination event in one allele of the p27 gene. To generate chimeras 6 of the p27^{+/-} ES cell clones were injected individually into blastocysts prepared from C57BL/6 females and transplanted the injected blastocysts to pseudo pregnant females. Three of the clones transmitted to the germ-line and from these, p27^{+/-} hybrid (C57BL/6×129Sv) mice (FIG. 1B) were generated. Further intercross of the p27^{+/-} mice produced offspring at predicted Mendelian ratios. Therefore, this targeted mutation of p27 does not affect survival during embryogenesis.

The cyclin/CDK inhibition domain was targeted for gene disruption rather than a complete deletion. To examine if the p27 mutant allele was capable of expressing any protein fibroblasts were prepared from embryos and analyzed the lysates by immunoblotting (FIG. 1C). Using an antibody to either full-length p27 or the carboxyl-terminus of p27 a 20 kDa protein in p27^{-/-} fibroblasts, and a 27 kDa protein in p27^{+/+} fibroblasts were detected. Both proteins were detected in p27^{+/31} fibroblasts (data not shown). In contrast, antibody specific to the amino-terminus of p27 recognized only the 27 kDa protein and not the 20 kDa protein (FIG. 1C). The amount of the 20 kDa species and p27 were comparable. Using PCR, RNA transcripts from the p27 mutant allele were amplified and its structure determined; the mutant transcript had the insertion of the neo gene in the antisense direction and normal splicing of the intron separating exons I and II. This transcript contains a predicted open reading frame that encodes amino acid 52-198 of the p27 protein (data not shown).

Taken together, the disruption of the p27 gene produced an amino-truncated mutant of p27 protein which was called Δ51. To test the function of Δ51 as an inhibitor of CDKs, the His tagged Δ51 from bacteria was purified and added it to extracts of Sf9 cells coinfected with baculoviruses expressing either cyclin E and CDK2, or cyclin D2 and CDK4, and measured the Rb kinase activity of the cyclin/CDK complexes. Δ51 inhibited Rb-phosphorylation by these kinases less efficiently than the full length p27 (FIG. 1D). Furthermore, Δ51 interacted poorly with cyclin/CDK complexes in cells (FIG. 1C). These results suggest that at the amounts produced in cells, Δ51 will not inhibit G1 CDKs and will not act in a dominant negative fashion to exclude the binding of other CDK inhibitors.

Mice Lacking Functional p27 are Larger Than Normal

Following weaning (day 21 to day 28) the p27^{-/-} mice weighed 20-40% more than sex matched littermate controls. A representative litter of male mice at 8 weeks of age is shown in FIG. 2A. A comparison of male mice, derived exclusively from intercross breeding, suggested that the weight of the mouse was a function of the p27 gene copy number (FIG. 2B). Analysis of pups from intercross breeding at the first three weeks post-partum indicated that the mean weight of p27^{-/-} mice was generally higher than p27^{+/+} mice, but there was significant overlap in the weight of each population. In 8 week old mice the difference between the populations was much greater and the smallest p27^{-/-} mice weighed as much as the largest p27^{+/+} mice (p<0.01). The mean weight and range of p27^{+/-} mice were intermediate. To determine the earliest time after birth that could be detected this difference, the mice were weighed from one week post-partum. From a total of 252 offspring, there were 21 p27^{-/-} mice in litters where sex matched comparisons to controls could be made. Although many factors might influence the weight gain in animals during nursing, such as competition between litter mates and the nutritional state of the mother, most of the p27^{-/-} mice (71%, n=15) were larger than littermate p27^{+/+} controls during the first 14 days post-partum (data not shown). However, of the remaining mice (29%, n=6) whose weight was either less than or equal to their wild-type siblings within the first 14 days, by day 32 post-partum they weighed more. Representative growth curves of two pairs of sex matched siblings are shown in FIG. 2C.

To determine if there was a correlation between weight and growth, skeletal growth and organ weight were examined. To evaluate skeletal growth, bone lengths radiographically (n=5) were determined. Differences in the length of the skull and longitudinal bones including the femur, tibia, and humerus that corresponded to the increase in the size of the mouse (data not shown) were observed. To examine if enlargement of visceral organs was also proportional to that of the mice, wet weights of the brain, thymus, heart, liver, spleen, kidney, and of the carcass from which the organs were removed were measured. Organ weights were consistently greater in p27^{-/-} mice as compared to controls, but did not invariably increase in proportion to the weight of the animal (FIG. 3A). The weight of the thymus and spleen increased the most, relative to the increase in body weight. Carcass weight increased proportionally to body weight suggesting that increases in the growth of muscle and/or connective tissue are partially responsible for the increased body weight (data not shown).

Since the effect of the p27 mutation on the weight of various organs varied in relation to the total body weight, it is speculated that the expression and function of p27 might be tissue-specific. The expression of wild-type and mutant p27 protein in the organs of p27^{+/+} and p27^{-/-} mice were therefore analyzed, respectively (FIG. 3B). While all p27^{+/+} tissues had detectable amounts of p27 protein, it was most abundant in thymus and spleen, consistent with the observation that the weight of these organs was the most affected by p27 gene disruption. The expression of the truncated mutant protein in p27^{-/-} mice showed a similar, although not identical, tissue-specificity (FIG. 3B). The biologic significance of this was not pursued in these studies. Loss of p27 did not affect tissue-specific expression of the other Kip family proteins, p21 and p57 (data not shown).

Lack of Functional p27 Leads to Hyperplasia in the Intermediate Lobe of the Pituitary

Transgenic mice expressing GH (Palmiter et al., 1982) or IGF-I (Mathews et al., 1988) grow larger than control mice. GH, secreted from the pituitary in response to hypothalamic signals, regulates postnatal body growth mainly by stimulating IGF-I expression in the peripheral tissues. To determine if the size of the p27^{-/-} mice was caused by changes of the GH/IGF-I axis the effect of p27 gene disruption on the pituitary was investigated. All the mice were examined at 11 weeks of age (n=6). The pituitary gland appeared increased in size with a prominent midline intermediate-posterior lobe region. Standard H & E staining revealed intermediate lobe hyperplasia with normal cellular morphology and arrangement, and normal vascularity (FIG. 4A, middle panel). The anterior lobes appeared compressed, with increased cellular density, while the posterior lobe appeared normal. In 30 week old mice (n=3) the intermediate lobe was even more hypercellular, with circular nests of cells giving the appearance of nodules (FIG. 4B, right panel). A marked increase in vascularity was present throughout the lobe, manifested as lakes of distended capillaries filled with red blood cells. Cellular morphology, however, was normal and no evidence of tumor formation was present. The anterior lobes were even more compressed than at 11 weeks. In some animals the tissue mass was sufficiently large as to cause compression of the ventral hypothalamus. The intermediate lobe contains cells that produce alpha-MSH. There was homogeneous staining with an antibody that reacts with POMC-derived peptides, including alpha-MSH, in the IL cells at 11 weeks (FIG. 4B). At 30 weeks, however, the staining was non-homogeneous with some of the nodular regions exhibiting intense staining while in others, staining was markedly decreased, in some, to the point of non-staining.

To determine if the IL hyperplasia affected the GH/IGF-I axis, immunohistochemistry was used to detect GH. The somatotropes producing GH are in the anterior lobe of the pituitary. In 11 and 30 week old p27^{-/-} mice, both the numbers of somatotropes and the intensity of GH staining were comparable to controls (data not shown). The direct assessment of GH secretion from single measurements of serum GH level is difficult because of the pulsatile nature of secretion and the influence of stress. IGF-I is the major GH dependent post-natal growth factor. IGF-I induces cell proliferation in major body components including the bone, cartilage, and connective tissue. It was next determined if the serum level of IGF-I was perturbed in p27^{-/-} mice. Applicants did not find any significant difference in serum levels of IGF-I. The amount of IGF-I in 27 samples isolated from six pairs of animals as described in the methods section was measured. The level of IGF-I in sera isolated from both male and female p27^{+/+} mice was 36±12 ng/ml (mean+SEM) with values ranging from 19-63 ng/ml. In p27^{-/-} mice, both male and female, the IGF1 levels were 34±20 ng/ml with values ranging from 5-64 ng/ml. When comparing measurements on a sex-matched basis, less variation in the amounts of serum IGF-I was found. These results indicate that the enhancement of postnatal body growth in the p27^{-/-} mice is independent of the regulation by the GH/IGF-I hormonal system.

p27 Disruption Increases the Number of S-phase Thymocytes

The disruption of p27 might affect the proportion of proliferating cells in a tissue before maturation. In most specialized cell types of the animal the proliferative index decreases following birth; however, thymocytes are still in the process of expansion and can be used to determine if the absence of p27 affected the number of proliferating cells. An increase in the number of splenic T-cells in p27^{-/-} mice (Table I) was detected.

An increase in T-cell number might occur either following antigen exposure in the periphery or during maturation in the thymus. To determine if peripheral T-cells lacking p27 were more sensitive to antigen, splenic T-cells were activated by cross-linking the T-cell receptor with increasing amounts of antibody to CD3. For each amount of anti-CD3 added, the activation of lymphocytes isolated from the p27^{-/-} mouse was equivalent to the control (data not shown).

To determine if the p27^{-/-} genotype affected thymocyte maturation the proportion of thymocytes at various stages of T-cell development in mice (n=8) was examined by fluorescence activated cell sorting (FACS). The values indicated in the table represent the mean of three 5-8 week old female mice, because the total thymocyte number was variable between animals at different ages and of different sexes. In these thymocyte populations the number of cells at each stage was greater in p27^{-/-} mice than in controls, although the percentage of cells was approximately equivalent with the possible exception of the CD44⁺CD25⁻ cells (Table I). In one 8 week old mouse, the percentage of CD44⁺CD25⁻ cells was increased two-fold, although the other thymocyte sub-populations were not affected. CD44⁺CD25⁻ cells represent the earliest thymocyte stage examined and the biological significance of this result is not clear.

Thymocyte number is a function of the balance between cell proliferation and cell death. To determine if p27 affected proliferation, death, or both, these processes in the thymocytes of p27^{-/-} mice and controls were examined. To detect thymocytes engaged in S-phase, four-week old animals were injecte with a single intraperitoneal injection of BrdU, and following dissection two hours later, the extent of BrdU incorporation into chromosomal DNA was measured by immunohistochemistry. It was found an increase in the number of BrdU-positive thymocytes in thymus from p27^{-/-} animals, in both the cortical region (FIG. 5A, panels b and e) (largely populated by immature thymocytes) and the medullary region (FIG. 5A, panels a and d) (largely populated by mature thymocytes). Moreover, this increase in the number of BrdU positive cells was greater than could be accounted for by the increase in cell number. The percentage of BrdU positive cells in random medullar fields of several sections was quantified. This analysis showed that the loss of p27 resulted in high levels of thymocyte proliferation. The mean percentage of BrdU-positive cells in three p27^{+/+} mice was 10% with values in random fields ranging from 3% to 19%. In contrast, the mean percentage of BrdU⁺ cells in three p27^{-/-} mice was 26% with values ranging from 24% to 33%. Furthermore, it was detected increased BrdU incorporation in both the cortical and medullar regions of the thymus suggesting that p27 gene disruption might affect the percentage of S-phase cells in multiple thymocyte sub-populations. It was also observed increased BrdU incorporation in the spleen (data not shown).

The sensitivity of isolated thymocytes to apoptosis-inducing conditions was next measured. Thymocytes from p27^{-/-} and control mice were exposed to either g-irradiation or dexamethasone. These treatments induced cell death in a similar percentage of p27^{-/-} and control thymocytes (FIG. 5B). Taken together these data suggest that loss of p27 might increase the proportion of cells engaged in the mitotic cycle.

Female Mice Lacking Functional p27 are Infertile

During the course of these experiments it was found that p27^{-/-} males could impregnate wild-type females; however, neither wild-type nor p27^{-/-} males could successfully impregnate p27^{-/-} females. Specific female infertility could result from a variety of defects intrinsic to the ovarian cells and/or of endocrine origin; the inability to produce cyclical changes of the appropriate magnitude in pituitary hormones such as follicle stimulating hormone (FSH) and luteinizing hormone (LH), failure to ovulate in response to changes in FSH and LH levels, intrinsic failure of the oocyte to be fertilized or develop, or failure to achieve an environment suitable for the development of the embryo. Histology, immunohistochemistry, and hormone treatment were used to address these possibilities.

To determine phases of the estrus cycle, the cytology of vaginal smears was examined. Vaginal smears taken from 8-20 week old mice showed that control mice passed though diestrus (day 1), proestrus (day 2), estrus (day 3), and metestrus (day 4) in four days (FIG. 6) as described previously (Nelson et al., 1982). In contrast, all the p27^{-/-} mice had prolonged estrus cycles, typically showing a prolongation prior to estrus and a delay in exiting estrus (FIG. 6). The most characteristic was the diestrus-proestrus like smear with numerous leukocytes and nucleated epithelial cells within abundant mucous secretion. This smear type often was present for 5-7 days. After a short transition, a prolonged estrus phase followed and persisted for 4-5 days. p27^{-/-} female mice (n=8) were capable of mating, albeit infrequently, as confirmed by formation of vaginal plugs; however, none of the mice maintained a pregnancy to the point where swelling of the abdomen could be observed. It was next examined if the mated females conceived. On day 3.5 after formation of the plug three month old females (n=3) were sacrificed and morula stage embryos were isolated from these mice indicating that fertilization had occurred. Furthermore, these morula developed to full-term when transferred to the oviducts of a pseudopregnant normal female. This suggests that ovulation and fertilization do occur in p27^{-/-} mice, albeit with decreased frequency, and oocyte development itself does not require p27.

The disordered estrus cycle might reflect an underlying problem in endocrine signaling between the pituitary and ovary. To determine if the ovaries of the p27^{-/-} mice would respond to exogenous gonadotropin stimulation similarly to controls; three-week old mice were injected with an FSH preparation, pregnant mare serum, and forty-eight hours later with an LH substitute, human chorionic gonadotropin (hCG) and observed the effect on ovulation. This treatment greatly exaggerates the levels and sequential nature of the endogenous hormones. Under these conditions it was confirmed ovulation in both p27^{+/+} and p27^{-/-} mice by microscopic analysis of cells flushed from the oviducts 24 hours after hCG injection.

The infertility of p27^{-/-} females might be due not only to irregular ovulation, but also to defects in the ability to maintain an environment suitable to maintain pregnancy. Corpus luteum formation plays an important role in maintenance of pregnancy by actively secreting progesterone and other factors. Granulosa cells, the major somatic cell component of follicles differentiate, by a poorly defined pathway, into progesterone producing luteal cells following ovulation. To determine if p27 was involved in luteal cell differentiation the expression of p27 was examined by immunohistochemistry. In control mice, p27 protein was undetectable in the granulosa cells of the follicle but was abundant in the cells of the corpus luteum (FIG. 7A, left panel). It was observed a reciprocal pattern of BrdU staining—incorporation was highest in the follicular granulosa cells and lowest in luteal cells (FIG. 7A, middle panel). Control sections in which primary antibody was omitted were uniformly negative (FIG. 7A, right panel). Examination of p27^{-/-} ovaries indicated a disruption in formation of corpus luteum (FIG. 7B). These data suggest that the absence of p27 might affect the transition from proliferating granulosa cell to non-proliferating luteal cell.

### Experimental Discussion

p27 might have a central function to control of cell proliferation and in turn to animal growth. In animals, both complex regulatory signaling networks between cell types and intrinsic responses of cells to particular signals determine phenotype. Here it is reported that loss of p27 function as an inhibitor of G1 cyclin-dependent kinases affects the response of cells to environmental signals resulting in increased body growth. p27 disruption also appears to alter endocrine signaling by affecting the decision of the cells involved in the hypothalamic-pituitary-ovarian axis to proliferate or withdraw from the mitotic cycle.

The function of p27, as a CDK inhibitor, is incompletely understood. Evidence from previous studies suggests that p27 might establish an inhibitory threshold, set in part by mitogenic and anti-mitogenic signaling, which G1 CDKs must surpass before activation and entry into S-phase (Sherr and Roberts, 1995; Koff and Polyak, 1995). In a number of cell types there is a correlation between growth arrest and increases of the CDK inhibitory activity of p27. Introduction of p27 to proliferating cells by transfection with appropriate vectors rapidly leads to growth arrest in G1 (Polyak et al., 1994b; Toyoshima and Hunter, 1994), and antisense induced reduction of p27 increases the proportion of S-phase cells (Coats et al., 1996). These properties make it attractive to speculate that p27 might be, at least in part, responsible for establishing the balance between proliferating and non-proliferating cells. For these reasons, it was decided to examine the role of p27 in proliferation and differentiation using a mouse model.

Disruption of p27 Affects the Manner in which Cells Respond to Extracellular Signals to Proliferate or Withdraw vfrom the Cell Cycle

As part of an inhibitory threshold, p27 might antagonize the S-phase promoting effect of CDKs and affect the decision to proliferate or withdraw from the mitotic cycle. In animals, the exposure of a cell to mitogenic and anti-mitogenic signals is rarely an all-or-none event, though the cell makes an irrevocable choice in response to these signals. Thus, at each division a cell has a stochastic probability of withdrawing from the mitotic cycle, determined by the nature of environmental signals: the loss of p27 might affect how cells respond. Mitogens and anti-mitogens induce both rapid and slow responses in cells. Exposure of either HL60 cells to TPA (Jiang et al., 1994; Steinman et al., 1994; J. S. Yan, HK, and AK, unpublished observations) or Mv1Lu cells to TGFb (Ewen et al., 1993; Koff et al., 1993; Slingerland et al., 1994; Reynisdottir et al., 1995) rapidly induce cyclin-dependent kinase inhibitors, and more slowly, a decrease in the amount of cyclin-dependent kinase proteins. If these signaling pathways target both events, the loss of p27 might not qualitatively prevent withdrawal from the mitotic cycle, but would affect the strength of the response. Thus, it is interpreted some of the phenotypes of the p27^{-/-} mice as a reduction in the capability of cells to respond to outside signals, either mitogenic or anti-mitogenic, consistent with an inhibitory threshold role based on the biochemical properties of p27.

It is found that disruption of p27 affects the balance between proliferating and non-proliferating cells in the animal. There is an increase in the fraction of mitotic cells in the thymus and spleen isolated from young mice. This effect of p27 gene disruption might be largely restricted to tissues undergoing post-natal maturation and reconstitution. Further analysis during embryogenesis will be required to address this. In addition, the expression of p27 correlates with differentiation of luteal cells. A well-developed corpus luteum were unable to be detected in p27^{-/-} mice; however, one cannot exclude the possibility that this is due to either hormonal changes, the loss of p27 in differentiating cells, or a combination of both. There is little known about the transition from granulosa cell to luteal cell and the cells without p27 might either be trapped in the cell cycle and unable to differentiate, withdrawn from the cell cycle but unable to differentiate, or undergoing apoptosis. In contrast, most developmental programs in the p27^{-/-} mouse do succeed normally (or nearly so) since specialized organs form and mice are viable. This suggests that p27 might regulate withdrawal from the mitotic cycle dependent on the type of signals the cell receives, and the type of redundant response the signal might elicit in the cell. These observations are consistent with previous studies demonstrating that the CDK-inhibitory activity of p27 correlated with G1-arrest (Polyak et al., 1994b; Toyoshima and Hunter, 1994), and that modulation of the amount of p27 can alter the proportion of cells engaged in the mitotic cycle (Coats et al., 1996).

Hyperplasia of the Intermediate Lobe of the Pituitary Suggests that p27 Might Maintain Cells in a Non-proliferative State

The disruption of p27 gives rise to intermediate lobe pituitary hyperplasia without evidence of adenoma (up to 30 weeks). Although all cells are originally alpha-MSH positive the staining in the hyperplastic nodules is variable. It is speculated that p27 might be required to maintain these cells in a non-proliferative differentiated state. The significance of hyperplasia restricted to these cells is unclear. Mice that have lost heterozygosity at the retinoblastoma (Rb) locus develop adenoma of the intermediate lobe (Jacks et al., 1992; Hu et al., 1994). Perhaps these cells are exquistely sensitive to the loss of negative regulators of cell cycle progression. One possibility is that these highly differentiated cells might retain the capacity to undergo post-natal reconstitution. In addition, this observation suggests that p27 and Rb might be in the same genetic pathway regulating proliferation of these cells. This is consistent with the findings that CDK phosphorylation of Rb decreases its ability to suppress growth (Hinds et al., 1992). Hyperplasia, rather than adenoma, might occur in p27^{-/-} mice because of either redundancy of regulation at the level of CDK activity, or the loss of the p27-inhibitory pathway might not be sufficient to obviate all the properties of Rb that make it a tumor suppressor.

Female Fertility Defects Implicate Changes in the Nature of Endocrine Signaling in p27^{-/-} mice

Alteration in the fate of cell proliferation might affect signaling between organs. A complex temporal hormonal network regulates estrus involving hypothalamic gonadotropin releasing hormone (GnRH), pituitary FSH and LH from the gonadotrope, and sex steroids produced in the ovary. GnRH expression leads to gonadotrope release of FSH and LH. FSH interacts with ovarian granulosa and theca cells and induces expression of estrogen synthetic enzymes and LH receptors. Later during the estrus cycle, the LH receptor expressing granulosa cells respond to pulsatile LH secretion and ovulation follows a short time later. The remnant follicle composed of granulosa cells, in some undefined manner, subsequently undergoes differentiation into luteal cells that produce the estrogen and progesterone necessary to maintain pregnancy. If the oocyte is not fertilized, the corpus luteum disintegrates, and a new cycle begins. In addition, the products of other endocrine networks can modulate this network. It is interpreted that the estrus phenotype of the p27^{-/-} mouse to reflect perturbation of endocrine signaling networks between the ovary and the pituitary.

At this time, it is speculated that in p27^{-/-} mice signaling between the ovary and the pituitary is compromised, however further work is required to determine the mechanism by which IL hyperplasia, the perturbed estrus cycle, and luteal cell differentiation are linked. Pituitary hyperplasia might affect either GnRH, FSH, and/or LH production. Hyperplasia might interfere directly or indirectly with other endocrine networks that modulate the GnRH/FSH-LH axis. Perhaps more importantly, is the inability to form a corpus luteum and its potential effects on endocrine pathways. Corpus formation is a signal of successful pregnancy, and luteal cells are responsible for production of estrogen and progesterone. In addition, other hormones, produced in the luteal cells, such as inhibin and relaxin, might regulate pituitary function. The prolonged estrus phase might be a reflection of corpus luteum dysfunction, because initiation of the next estrus cycle requires completion of the previous cycle. Completion is signaled by corpus formation followed either by pregnancy or corpus disintegration. Consequently, the endocrine defect might be, at least in part, due to the inability of granulosa cells to properly differentiate into luteal cells, although the involvement of the pituitary as a primary factor cannot be ruled out by our observations.

Enhanced Growth of Mice with Disrupted p27 Alleles Reflects its Function as an Intracellular Regulator of Proliferation

Like ovulation, a complex endocrine network regulates growth, consisting of hypothalamic growth hormone releasing hormone (GHRH) and somatostatin, positive and negative regulators of the somatotropes that secrete GH.

GH acts on liver cells to induce IGF-I production, and IGF-I increases protein synthesis and mitogenesis in target cells. Transgenic mice expressing either GH or IGF-I grow to a larger size than control animals (Palmiter et al., 1982; Mathews et al., 1988). However, our data indicate the GH/IGF-I hormonal axis does not play a major role in the p27^{-/-} growth phenotype. First, the number of somatotropes and the amount of stored GH appear identical in p27^{-/-} and control animals. Second, there is no increase in the level of serum IGF-I indicating that the responses by IGF-I producing cells are not enhanced to normal levels of GH. However, the proliferative response of p27^{-/-} cells to IGF-I might be enhanced.

It is speculated that the increase in mitotic cells before maturation of an organ might explain, in part, increased growth of the animal. First, p27 gene disruption results in an increase in the proportion of cycling cells and cell number. Second, the growth of p27^{-/-} mice was a function of p27 gene copy number and p27 protein expression. p27^{-/-} mice were significantly larger than p27^{+/-} mice, which were in turn larger than wild-type litter mates. The thymus and spleen, which in control animals express the highest amounts of p27 were significantly increased in weight whereas organs that normally express lower levels of p27 were less affected by its disruption. Third, it is observed that embryonic fibroblasts and splenic T-cells isolated from p27^{-/-} mice are more resistant to the anti-proliferative effects of rapamycin (Luo et al., submitted). Together these data suggest that p27 disruption might affect animal growth in part by altering the balance between proliferating and non-proliferating cells at critical periods of development which might vary for each organ.

It is interpreted our results to indicate that p27, as an inhibitor of G1 CDKs, affects growth of mice by increasing the proportion of cycling cells before maturation. It is speculated that p27 is a true regulator of growth by exerting its actions on the decision of a cell to either proliferate or withdraw from the cell cycle, in response to environmental signals. In single cell eukaryotes and cells of vertebrate origin, growth is an accumulation of sufficient cell mass (Prescott, 1976). However, growth of an animal is due, in part, to a net increase in the number of cells. Until now, there has not been a report of an intracellular regulator of animal growth. Accordingly, p27 might have a central function, albeit partially redundant, to control of cell proliferation and in turn to enhance animal growth.

### SECOND SERIES OF EXPERIMENTS

Culture of Hematopoietic Progenitors

To evaluate the proliferation activity of hematopoietic in the p27−/− mice, splenocytes from mice in methylcellulose medium supplemented with a variety of cytokines were cultured. Spleen cell suspensions were prepared from p27−/− or p27+/+ mice, and erythrocytes eliminated by incubation with hemolysis buffer. Equal numbers (6×10⁵) of the resulting splenocytes were plated into a 60-mm culture dish with 3 ml of alpha-minimum essential supplemented with 0.88% methylcellulose, 30% fetal calf serum, 1% bovine serum albumin, 0.1 mM 2-mercaptoethanol, 2 mM L-glutamine, 3 U/ml erythropoietin and 2% (v/v) PWM-stimulated murine spleen cell conditioned medium. Conditioned medium is a source of cytokines including stem cell factor (kit-ligand), IL-3 and GN-CSF. After 12 days of culture, at 37° C. in a 5% CO₂ atmosphere, colonies of erythroid (BFU-E), granulocytE!/macrophage (CFU-GM) and mixed pluripotent (CFU-GEMM) lineages were scored.

It was observed a significant increase in the number of granulocyte/macrophage progenitors and mixed pluripotent progenitors in the spleens isolated from p27−/− mice (Table 1). This observation was confirmed in three independent experiments. However, there was extensive variation in the number of erythroid colonies and it was not conclusive whether there is an increase in erythroid progenitors.

TABLE 1
Hematopoietic stem cells

| Genotype | BFU-E | CFU-GM | CFU-GEMM |
|---|---|---|---|
| p27−/− | 13.5 + 14.8 | 80.5 + 43.13 | 5.5 + 0.7 |
| p27+/+ | 6.0 + 1.4 | 20.5 + 0.7 | 1.5 + 0.7 |

It was also observed that there is a significant increase in the number of thymocytes in the p27−/− as compared with the wild type animal (table 2).

TABLE 2
Comparison of thymocyte cell number in p27−/− and p27+/+.

| | | | fold increase in | | |
|---|---|---|---|---|---|
| | Cell Number | | cell number | Percent of | |
| | (× 10⁶) | | attributed to p27 | population | |
| Cell Type | +/+ | −/− | gene disruption | +/+ | −/− |
| Total | 34.20 | 63.57 | 1.9 | | |
| Spenic | | | | | |
| T-cells | | | | | |
| Total | 176.67 | 480.00 | 2.7 | | |
| Thymocytes | | | | | |
| CD44+ | 0.33 | 1.51 | 3.1 | 0.19 | 0.31 |
| CD25− | | | | | |
| CD44+ | 0.08 | 0.25 | 2.6 | 0.05 | 0.05 |
| CD25+ | | | | | |
| CD44lo | 1.62 | 4.18 | 2.6 | 0.88 | 0.78 |
| CD25+ | | | | | |
| CD44lo | 1.87 | 4.89 | 2.7 | 1.06 | 1.02 |
| CD25− | | | | | |
| CD4+ CD8+ | 154.27 | 423.03 | 2.5 | 87.30 | 88.11 |
| CD4+ CD8− | 13.54 | 33.75 | 2.5 | 7.68 | 7.03 |
| CD4− CD8+ | 4.98 | 12.53 | 2.7 | 2.82 | 2.61 |

The Steady-state Number of Thymocytes is Higher in p27−/− Animals than Controls

In order to determine if thymocyte maturation reached a steady-state in p27−/− mice, the total number of thymocytes isolated from both male and female 5, 8, and 13 week old mice as applicants previously described (Kiyokawa, et al. 1996 Cell, In Press) were counted. Preliminary analysis is described in table 3. Five-week old male mice have a significantly greater number of thymocytes than female mice restricting analysis to a sex matched basis.

TABLE 3
Thymocytes number as a function of sex and age.

| p27+/+ | | | | P27−/− | | | |
|---|---|---|---|---|---|---|---|
| | | | number of | | | | number of |
| age | | | thymocytes | age | | | thymocytes |
| (weeks) | sex | n | (10⁶) | (weeks) | sex | n | (10⁶) |
| 5 | M | 3 | 256 +/− 64 | 5 | M | 3 | 579 +/− 109 |
| 5 | F | 1 | 132 | 5 | F | 1 | 224 |
| 8 | F | 2 | 199 +/− 50 | 8 | F | 2 | 608 +/− 111 |
| 13 | F | 1 | 160 | 13 | F | 1 | 755 |

It is found that the number of thymocytes varies little with age of control mice; however, the number of thymocytes increases with age in the p27−/− mice (FIG. 8).

### REFERENCES

Chen, I.-T., Akamatsu, M., Smith, M. L., Lung, F.-Di. L., Duba, D., Roleer, P. P., Fornace, A. J. Jr., and O'Connor, P. M. (1996) Characterization of p21 Cip1/Waf1 peptide domains required for cyclin E/Cdk2 and PCNA interaction. Oncogene 12, 595-607.

Clarke, A. R., Purdie, C. A., Harrison, D. J., Morris, R. G., Bird, C. C., Hooper, M. L., and Wylie, A. H. (1993) Thymoycte apoptosis induced by p53-dependent and independent pathways. Nature 362, 849-852.

Coats, S., Flannagan, W., Nourse, J., and Roberts, J. M. (1996) Science, in press.

Dudley, E. C., Petrie, H. T., Shah, L. M., Owen, M. J., and Hayday, A. C. (1994) T cell receptor b chain gene rearrangment and selection during thymocyte development in adult mice. Immunity 1, 83-93.

Dulic, V., Kaufmann, W. K., Wilson, S. J., Tlsty, T. D., Lees, E., Harper, J. W., Elledge, S. J., and Reed, S. I. (1994) p53-dependent inhibition of cyclin-dependent kinase activities in human fibroblasts during radiation-induced G1 arrest. Cell 76, 1013-1023.

Ewen, M. E., Sluss, H. K., Whitehouse, L. L., and Livingston, D. M. (1993). TGFb inhibition of CDK4 synthesis is linked to cell cycle arrest. Cell 74, 1009-1020.

Geng, Y., and Weinberg, R. A. (1993). Transforming growth factor b effects on expression of G1 cyclins and cyclin-dependent protein kinases. Proc. Natl. Acad. Sci. USA 90, 10315-10319.

Gu, Y., Turck, C. W., and Morgan, D. O. (1993). Inhibition of CDK2 activity in vivo by an associated 20K regualtory subunit. Nature 366, 707-710.

Guan, K. L., Jenkins, C. W., Li, Y., Nichols, M. A., Wu, X., O'Keefe, C. L., Matera, A. G., and Xiong, Y. (1994). Growth suppression by p18, a p16INK4/MTS1- and p14INK4B/MTS2-related CDK6 inhibitor correlates with wild-type Rb function. Genes & Dev. 8, 2939-3952.

Guan, K. L., Jenkins, C. W., Li, Y., O'Keefe, C. L., Noh, S., Wu, X., Zariwala, M., Matera, A. G., and Xiong, Y. (1996). Isolation and characterization of p19INK4d, a p16-related inhibitor specific to CDK6 and CDK4. Mol. Biol. Cell 7, 57-70.

Gyuris, J., Golemis, E., Chertkov, H., and Brent, R. (1993). Cdil, a human G1 and S phase protein phosphatase that associates with Cdk2. Cell 75, 791-803.

Hannon, G. J., and Beach, D. (1994) p15INK4B is a potential effector of TGFb-induced cell cycle arrest. Nature 371, 257-261.

Harper, J. W., Adami, G. R., Wei, N., Keyomarsi, K., and Elledge, S. J. (1993). The p21 Cdk-interacting protein Cip1 is a potent inhibitor of G1 cyclin-dependent kinases. Cell 75, 805-816.

Harper, J. W., Elledge, S. J., Keyomarsi, K., Dynlacht, B., Tsai, L. H., Zhang, P., Dobrowolski, S., Bai, C., Connell-Crowley, L., Swindell, E., Fox, M. P., andwei, N. (1995). Inhibition of cyclin-dependent kinases by p21. Mol. Biol. Cell 6, 387-400.

Hinds, P. W., Mittnacht, S., Dulic, V., Arnold, A., Reed, S. I., and Weinberg, R. A. (1992) Regulation of retinoblastoma functions by ectopic expression of human cyclins. Cell 70, 993-1006.

Hu, N., Gutsmann, A., Herbert, D. C., Bradley, A., Lee, W. H., and Lee, E. Y. -H. P. (1994). Heterozygous Rb-1-^{20/+} mice are predisposed to tumors of the pituitary gland with a nearly complete penetrance. Oncogene 9, 1021-1027.

Jacks, T., Fazeli, A., Schmitt, E. M., Bronson, R. T., Goodell, M. A., and Weinberg, R. A. (1992). Effects of an Rb mutation in the mouse. Nature 359, 295-300.

Jiang, H., Lin, J., Su, Z. Z., Collart, F. R., Hubermann, E., and Fisher, P. B. (1994). Induction of differentiation in human promyelocytic HL-60 leukemia cells activates p21 WAF1/CIP1: expression in the absence of p53. Oncogene 9, 3397-3406.

Kato, J. Y., Matsuoka, M., Polyak, K., Massague, J., and Sherr, C. J. (1994) Cyclic AMP induced G1 phase arrest mediated by an inhibitor (p27KIP1) of cyclin-dependent kinase 4 activation. Cell 79, 487-496.

Koff, A., Ohtsuki, M.., Polyak, K., Roberts, J. M., and Massagué, J. (1993) Negative regulation of G1 in mammalian cells: Inhibition of cyclin E-dependent kinase by TGF-b. Science 260, 536-539.

Koff, A., and Polyak, K. (1995). p27KIP1, an inhibitor of cyclin-dependent kinases. In Progress in Cell Cycle Research Volume 1, L. Meijer, S. Guidet, and H. Y. L. Tung, eds (New York, USA: Plenum Press), pp.141-147.

Kranenburg, O., Scharnhorst, V., Van der Eb, A., and Zantema, A. (1995). Inhibition of cyclin-dependent kinase activity triggers neuronal differentiation of mouse neuroblastoma cells. J. Cell Biol. 131, 227-234.

Lee, M. H., Reynisdottir, I., and Massague, J. (1995). Cloning of p57^{KIP2}, a cyclin-dependent kinase inhibitor with a unique domain structure and tissue distribution. Genes & Dev. 9, 639-649.

Liu, M., Lee, M. H., Cohen, M., Bommakanti, M., and Freedman, L. P. (1996). Transcriptional activation of the Cdk inhibitor p21 by vitamin D3 leads to the induced differentiation of the myelomonocytic cell line U937. Genes & Dev. 10, 142-153.

Lowe, S. W., Schmitt, E. M., Smith, S. W., Osborne, B. A., and Jacks, T. (1993) p53 is required for radiation-induced apoptosis in mouse thymocytes. Nature 362, 847-849.

Luo, Y., Hurwitz, J., and Massagué, J. (1995). Cell-cycle inhibition by independent CDK and PCNA binding domains in p21^{CIP1}. Nature 375, 159-161.

Mansour, S. L., Thomas, K. R., and Capecchi, M. R. (1988) Disruption of the proto-oncogene int-2 in mouse embryo-derived stem cells: a general strategy for targeting mutations to non-selectable genes. Nature 366, 348-354.

Mathews, L. S., Hammer, R. E., Behringer, R. R., D'Ercole, A. J., Bell, G. I., Brinster, R. L., and Palmiter, R. D. (1988). Growth enhancement of transgenic mice expressing human insulin-like growth factor I. Endocrinology 123, 2827-2833.

Matsuoka, S., Edwards, M. C., Bai, C., Parker, S., Zhang, P., Baldini, A., Harper, J. W., and Elledge, S. J. (1995). p57^{KIP2}, a structurally distinct member of the p21^{CIP1} Cdk inhibitor family, is a candidate tumor suppressor gene. Genes & Dev. 9, 650-662.

Matsushime, H., Quelle, D. E., Shurtleff, S. A., Shibuya, M., Sherr, C. J., and Kato, J.-Y. (1994) D-type cyclin-dependent kinase activity in mammalian cells. Mol. Cell. Biol. 14, 2066-2076.

Morstyn, G., Hsu, S. M., Kinsella, T., Gratzner, H., Russo, A. and Mitchell, J. B. (1983) Bromodeoxyuridine in tumors and chromosomes detected with a monoclonal antibody. J. Clin. Invest. 72, 1877-1883.

Nakanishi, M., Robetoyre, R. S., Adami, G. R., Pereira-Smith, O. M., and Smith, J. R. (1995). Identification of the active region of the DNA synthesis inhibitory gene p21^{Sdi1/CIP1/WAF1}. EMBO J. 14, 555-563.

Nelson, J. F., Felicio, L. S., Randall, P. K., Sims, C., and Finch, C. B. (1982) A longitudinal study of estrous cyclicity in aging C57BL/6J mice: I. Cycle frequency, length, and vaginal cytology. Biol. Reprod. 27, 327-339.

Ohtsubo, M., and Roberts, J. M. (1993). Cyclin-dependent regulation of G1 in mammalian fibroblasts. Science 259, 1908-1912.

Pagano, M., Tam, S. W., Theodoras, A. M., Beer-Romero, P., Del Sal, G., Chau, V., Yew, P. R., Draetta, G. F., and Rolfe, M. (1995). Role of the ubiquitin-proteasome pathway in regulating abundance of the cyclin-dependent kinase inhibitor p27. Science 269, 682-685.

Palmiter, R. D., Brinster, R. L., Hammer, R. E., Trumbauer, M. E., Rosenfeld, M. G., Brinberg, N. C., and Evans, R. M. (1982). Dramatic growth of mice that develop from eggs microinjected with metallothionein-growth hormone fusion genes. Nature 300, 611-615.

Pardee, A. B. (1989). G1 events and the regulation of cell proliferation. Science 246, 603-608.

Peter, M., and Herskowitz, I. (1995). Joining the complex: cyclin-dependent kinase inhibitory proteins and the cell cycle. Cell 79, 181-184.

Polyak, K., Kato, J. Y., Solomon, M. J., Sherr, C. J., Massagué, J., Roberts, J. M., and Koff, A. (1994a). p27^{KIP1}, a cyclin-Cdk inhibitor, links transforming growth factor-b and contact inhibition to cell cycle arrest. Genes & Dev. 8, 9-22.

Polyak, K., Lee, M. H., Erdjument-Bromage, H., Koff, A., Roberts, J. M., Tempst, P., and Massagué, J. (1994b). Cloning of p27^{KIP1}, a cyclin-dependent kinase inhibitor and a potential mediator of extracellular anti-mitogenic signals. Cell 78, 59-66.

Pardee, A. B. (1989). G1 events and the regulation of cell proliferation. Science 246, 603-608.

Prescott, D. M. (1976). Reproduction of eukaryotic cells. (New York, New York: Academic Press).

Quelle, D. E., Ashmun, R. A., Shurtleff, S. A., Kato, J. Y., Bar-Sagi, D., Roussel, M. F., and Sherr, C. J. (1993). Overexpression of mouse D-type cyclins accelerates G1 phase in rodent fibroblasts. Genes & Dev. 7, 1559-1571.

Resnitzky, D., Gossen, M., Bujard, H., and Reed, S. I. (1994). Acceleration of the G1/S phase transition by expression of cyclins D1 and E with an inducible system. Mol. Cell. Biol. 14, 1669-1679.

Resnitzky, D., and Reed, S. I. (1995). Different roles for cyclin Di and E in regulation of the G1-to-S transition. Mol. Cell. Biol. 15, 3463-3469.

Reynisdottir, I., Polyak, K., Iavarone, A., and Massagué, J. (1995). Kip/Cip and Ink4 Cdk inhibitors cooperate to induce cell cycle arrest in response to TGF-b. Genes & Dev. 9, 1831-1845.

Serrano, M., Hannon, G. J., and Beach, D. (1993). A new regulatory motif in cell cycle control causing specific inhibition of cyclin D/CDK4. Nature 366, 704-707.

Sherr, C. J. (1993). Mammalian GI cyclins. Cell 73, 1059-1065.

Sherr, C. J., and Roberts, J. M. (1995). Inhibitors of mammalian G1 cyclin-dependent kinases. Genes & Dev. 9, 1149-1163.

Shou, Wenying, and Dunphy, W. G. (1996). Cell cycle control by Xenopus p28^{Kix1}, a developmentally regulated inhibitor of cyclin-dependent kinases. Mol. Biol. of the Cell. 7, 457-469.

Slingerland, J. M., Hengst, L., Pan, C. H., Alexander, D., Stampfer, M. R., and Reed, S. I. (1994). A novel inhibitor of cyclin-Cdk activity detected in transforming growth factor b-arrested epithelial cells. Mol. Cell. Biol. 14, 3683-3694.

Soos, T. J., Kiyokawa, H., Yan, J. S., Rubin, M. S., Giordano, A., DeBlasio, A., Bottega, S., Wong, B., Mendelsohn, J., and Koff, A. (1996). Formation of p27-CDK complexes during the human cell cycle. Cell Growth & Diff. 7, 135-146.

Steniman, R. A., Hoffman, B., Iro, A., Guillouf, C., Liebermann, D. A., and El-Houseini, M. E. (1994). Induction of p21 (WAF-1/CIP1) during differentiation. Oncogene 9, 3389-3396.

Swiatek, P. J., and Gridley, T. (1993) Perinatal lethality and defects in hindbrain development in mice homozygous for a targeted mutation of the zinc finger gene Krox20. Genes & Dev. 7, 2071-2084.

Tanquay, D. A., and Chiles, T. C. (1994). Cell cycle-specific induction of Cdk2 expression in B lymphocytes following antigen receptor cross-linking. Mol. Immunol. 31, 643-649.

Toyoshima, H., and Hunter, T. (1994). p27, a novel inhibitor of G1 cyclin-Cdk protein kinase activity is related to p21. Cell 78, 67-74.

Wilson, A., D'Amico, A., Ewing, T., Scollay, R., and Shortman, K. (1988) Subpopulations of early thymocytes. A cross correlation flow cytometric analysis of adult mouse Ly-2-L3T4 (CD8-CD4-) thymoyctes using eight different surface markers. J. Immunol. 140, 1461-1470.

Xiong, Y., Hannon, G. J., Zhang, H., Casso, D., Kobayashi, R., and Beach, D. (1993). p21 is a universal inhibitor of cyclin kinases. Nature 366, 701-704.

Zhang, H., Hannon, G. J., and Beach, D. (1994). p21-containing cyclin kinases exist in both active and inactive states. Genes & Dev. 8, 1750-1758.

What is claimed is:

1. A method for increasing the proliferation of thymocytes in a non-human animal comprising altering an endogenous gene encoding p27^{Kip1} in a somatic cell of the animal to cause a functional deficiency of cyclin-dependent kinase inhibitor function of p27^{Kip1}, thereby increasing the proliferation of thymocytes in the animal.

2. The method of claim 1, wherein the cell is a thymocyte or bone marrow cell.

3. The method of claim 1, wherein the animal is a rodent, pig, sheep, frog, or bovine.

4. The method of claim 1, wherein the gene encoding p27^{Kip1} is altered by insertion of a positively selectable marker, mutation of the gene encoding p27^{Kip1}, or deletion of the gene encoding p27^{Kip1}.

5. The method of claim 4, wherein the gene encoding p27^{Kip1} is altered by insertion of a positively selectable marker into the gene.

6. The method of claim 5, wherein the positively selectable marker encodes neomycin resistance, thymidine kinase, adenine phosphoribosyl transferase, hypoxanthine-guanine phosphoribosyl transferase or dihydrofolate reductase.

7. The method of claim 6, wherein the positively selectable marker encodes neomycin resistance.

8. The method of claim 1, further comprising:
introducing a plasmid into the cell, wherein the plasmid comprises the gene encoding p27^{Kip1} altered by insertion of a positively selectable marker.

9. The method of claim 8, wherein the plasmid further comprises a negatively selectable marker adjacent the altered gene encoding p27^{Kip1}, whereby the distance between the negatively selectable marker and the altered gene encoding p27^{Kipl} is sufficient to allow homologous recombination between the altered gene encoding p27^{Kip1} and a gene encoding p27^{Kip1} in the cell.

10. The method of claim 9, wherein the negatively selectable marker encodes thymidine kinase.

11. The method of claim 8, wherein the plasmid is delivered to the cell by electroporation, microinjection or transformation.
